(19) United States
(12) Patent Application Publication
(10) Pub. No.: US 2015/0045305 A1
(43) Pub. Date: Feb. 12, 2015

(54) COMBINATION THERAPIES USING LATE SODIUM ION CHANNEL BLOCKERS AND POTASSIUM ION CHANNEL BLOCKERS

(71) Applicant: GILEAD SCIENCES, INC., FOSTER CITY, CA (US)

(72) Inventors: Luiz Belardinelli, Palo Alto, CA (US); Sridharan Rajamani, Newark, CA (US)

(21) Appl. No.: 14/374,467

(22) PCT Filed: Jan. 25, 2013
(86) PCT No.: PCT/US13/23291

§ 371 Date: Jul. 24, 2014

Related U.S. Application Data
(60) Provisional application No. 61/591,734, filed on Jan. 27, 2012.

(51) Int. Cl. A61K 38/17; A61K 31/553

(52) U.S. Cl. CPC A61K 38/1767; A61K 31/553
USPC 514/17.4; 514/211.05

(57) ABSTRACT

Described herein is a method for the treatment or prevention of atrial fibrillation and/or atrial flutter comprising administration of an effective amount of one or more of a potassium channel blocker and an effective amount of one or more of a late sodium channel blocker. Also provided are methods for modulating ventricular and atrial rhythm and rate. Also provided are pharmaceutical formulations that are suitable for such combined administration.

### CROSS REFERENCE TO RELATED APPLICATIONS

This application claims the benefit under 35 U.S.C. §119(e) of U.S. Provisional Application No. 61/591,734 filed on Jan. 27, 2012, which is hereby incorporated by reference in its entirety.

### FIELD

The present disclosure relates to methods of treating and/or preventing atrial fibrillation and/or atrial flutter by administration of an effective amount of one or more late sodium ion channel blocker and one or more potassium ion channel blocker. This disclosure also relates to pharmaceutical formulations that are suitable for such administration.

### BACKGROUND

Atrial fibrillation (AF) is the most prevalent arrhythmia, the incidence of which increases with age. It is estimated that 8% of all people over the age of 80 experience this type of abnormal heart rhythm and AF accounts for one-third of hospital admissions for cardiac rhythm disturbances. Over 2.2 million people are believed to have AF in the Unites States alone. Fuster et al Circulation 2006 114 (7): e257-354. Although atrial fibrillation is often asymptomatic it may cause palpitations or chest pain. Prolonged atrial fibrillation often results in the development of congestive heart failure and/or stroke. Heart failure develops as the heart attempts to compensate for the reduced cardiac efficiency while stroke may occur when thrombi form in the atria, pass into the blood stream and lodge in the brain. Pulmonary emboli may also develop in this manner.

Current methods for treating AF include electric and/or chemical cardioversion and laser ablation. Anticoagulants, such as warfarin, dabigatran, and heparin, are typically prescribed in order to avoid stroke. While there is currently some debate regarding the choice between rate and rhythm control, see Roy et al. N Engl J Med 2008 358:25; 2667-2677, rate control is typically achieved by the use of beta blockers, cardiac glycosides, and calcium channel blockers.

Ion channels are proteins that span the lipid bilayer of the cell membrane and provide an aqueous pathway through which specific ions such as Na⁺, K⁺, Ca²⁺ and Cl⁻ can pass. Herbert, Am. J. Med, 104, 87-98, 1998. Potassium channels represent the largest and most diverse sub-group of ion channels and they play a central role in regulating the membrane potential and controlling cellular excitability. Armstrong & Hille, Neuron, 20, 371-380, 1998. Potassium channels have been categorized into gene families based on their amino acid sequence and their biophysical properties.

Compounds which modulate potassium channels have multiple therapeutic applications in several disease areas including cardiovascular, neuronal, auditory, renal, metabolic and cell proliferation. Shieh et al. Pharmacol Rev 52(4), 557-594, 2000; Ford et al. Prog Drug Res, 58, 133-168, 2002. More specifically potassium channels such as Kv4.3, Kir2.1, HERG, KVLQT1/minK, and Kv1.5 are involved in the repolarisation phase of the action potential in cardiac atrial myocytes. These potassium channel subtypes have been associated with cardiovascular diseases and disorders including long QT syndrome, hypertrophy, ventricular fibrillation, and atrial fibrillation, all of which can cause cardiac failure and fatality. Marban, Nature, 415, 213-218, 2002.

Traditional and novel class III antiarrhythmic potassium channel blockers have been reported to have a mechanism of action by directly modulating Kv1.5 or Kv₍ᵤᵣ₎. The known class III antiarrhythmics ambasilide (Feng et al., J Pharmacol Exp Ther, 281(1), 384-392, 1997), quinidine (Wang et al., J Pharmacol, 272(1), 184-196, 1995.), clofilium (Malayev et al., Mol Pharmaco, 147(1), 198-205, 1995) and bertosamil (Godreau et al., J Pharmacol Exp Ther 300(2), 612-620, 2002) have all been reported as potassium channel blockers of Kv₍ᵤᵣ₎ in human atrial myocytes. The novel benzopyran derivative, NIP-142, blocks Kv1.5 channels, prolongs the atrial refractory period and terminates atrial fibrillation and flutter in in vivo canine models (Matsuda et al., Life Sci, 68, 2017-2024, 2001), and S9947 inhibited Kv1.5 stably expressed in both Xenopus oocytes and Chinese hamster ovary (CHO) cells and Kv₍ᵤᵣ₎ in native rat and human cardiac myocytes (Bachmann et al., Naunyn Schmiedebergs Arch Pharmacol, 364(5), 472-478, 2001). Elsewhere, other novel potassium channel modulators which target Kv1.5 or Kv₍ᵤᵣ₎ have been described for the treatment of cardiac arrhythmias, these include biphenyls (Peukert et al. J Med. Chem. February 13; 46(4):486-98, 2003), thiophene carboxylic acid amides (WO 2002/48131), bisaryl derivatives (WO 2002/44137, WO 2002/46162), carbonamide derivatives (WO 2001/00573, WO 2001/25189), anthranillic acid amides (WO 2002/100825, WO 2002/088073, WO 2002/087568), dihydropyrimidines (WO 2001/40231), cycloakyl derivatives (WO 2003/063797), indane derivatives (WO 2001/46155, WO 1998/04521), tetralin benzocycloheptane derivatives (WO 1999/37607), thiazolidone and metathiazanone derivatives (WO 1999/62891), benzamide derivatives (WO 2000/25774), isoquinoline derivatives (WO 2002/24655), pyridazinone derivatives (WO 1998/18475, WO 1998/18476), chroman derivatives (WO 1998/04542), benzopyran derivatives (WO 2001/21610, WO 2003/000675, WO 2001/21609, WO 2001/25224, WO 2002/064581), benzoxazine derivatives (WO 2000/12492), and the novel compound A1998 purified from Ocean material (Xu & Xu, Yi Chuan Xue Bao, 27(3), 195-201, 2000).

In addition to Kv1.5 or Kv₍ᵤᵣ₎ channels, additional potassium channels are also involved in treatment of AF. IKACh is an inwardly rectifying potassium channel that plays an important role in the regulation of mammalian heart rate. More specifically, IKACh is an acetylcholine-activated potassium current encoded by the Kir3.1/3.4 ion channel genes and is an emerging ion channel target for the development of new therapeutics for atrial fibrillation. IKACh is expressed only in the atria, and inhibition of IKACh is expected to maintain sinus rhythm in patients who have experienced episodes of atrial fibrillation. IKACh is activated by direct interaction with G betagamma subunits of pertussis toxin-sensitive heterotrimeric G-proteins.

The late sodium current (INaL) is a sustained component of the fast Na+ current of cardiac myocytes and neurons. Many common neurological and cardiac conditions are associated with abnormal INaL enhancement, which contributes to the pathogenesis of both electrical and contractile dysfunction in mammals. See, for example, Pathophysiology and Pharmacology of the Cardiac, Pharmacology and Therapeutics 119 (2008) 326-339. Accordingly, pharmaceutical compounds that selectively inhibit INaL in mammals are useful in treating such disease states.

One example of a selective inhibitor of INaL is RANEXA®, a compound approved by the FDA for the treatment of chronic angina. RANEXA® has also been shown to be useful for the treatment of a variety of cardiovascular diseases, including ischemia, reperfusion injury, arrhythmia and unstable angina, and also for the treatment of diabetes.

### SUMMARY

It is contemplated that by administering to a patient a late sodium channel blocker and a potassium channel blocker, a synergistic effect will be observed when treating atrial fibrillation and/or atrial flutter in patients, as well as a variety of other cardiac conditions, which are described throughout. It is further contemplated that the administration is useful when a potassium channel blocker is administered in an effective dose and a late sodium channel blocker is administered in an effective dose. It is further contemplated that either one or both of the potassium channel blocker and the late sodium channel blocker may be effective if being administered in an amount less than their respective effective doses when administered alone due to their synergistic effect.

Accordingly, in one aspect, the disclosure provides a method for treatment and/or prevention of atrial fibrillation and/or atrial flutter in a human patient in need thereof. The method comprises administration of an effective amount of one or more potassium channel blockers and an effective amount of late sodium channel blocker.

In another aspect is provided, a method for reducing undesirable side effects of a potassium channel blocker in a human patient in need thereof comprising administering an effective amount of one or more late sodium channel blockers.

In another aspect is provided, a method for reducing undesirable side effects of a late sodium channel blocker in a human patient in need thereof comprising administering an effective amount of one or more of a potassium channel blockers.

In another aspect is provided, a method for reducing a therapeutically effective dose of a potassium channel blocker comprising administering an effective amount of one or more late sodium channel blockers.

In another aspect is provided, a method for reducing a therapeutically effective dose of a late sodium channel blocker comprising administering an effective amount of one or more potassium channel blocker.

In another aspect is provided, a method for reducing prolongation of the QT interval in a human patient, said method comprising administering to the patient effective amounts of one or more potassium channel blockers and one or more late sodium channel blockers.

In another aspect is provided, a method for reducing prolongation of the QT interval in a human patient caused by potassium channel blocker or a late sodium channel blocker, said method comprising administering to the patient effective amounts of one or more potassium channel blockers and one or more late sodium channel blockers.

In another aspect is provided, a method for modulating ventricular and/or atrial rate in a human patient in need thereof, said method comprising administering to the patient effective amounts of one or more potassium channel blockers and one or more late sodium channel blockers. In one embodiment, AV conduction is slowed when atrial rate is high. In one embodiment, atrial rate is decreased. In another embodiment, heart rate is not significantly decreased during sinus rhythm.

In another aspect is provided, a method for modulating ventricular and/or atrial rhythm in a human patient in need thereof, said method comprising administering to the patient effective amounts of one or more potassium channel blockers and one or more late sodium channel blockers. In one embodiment, the sinus rhythm of the patient is maintained.

In another aspect is provided, a method for providing rhythm and rate control of the ventricles and/or atria in a human patient in need thereof, said method comprising administering to the patient effective amounts of one or more potassium channel blockers and one or more late sodium channel blockers. In aspects just described, the patient may optionally suffer from atrial fibrillation.

In another aspect is provided, a method for reducing or preventing torsades de pointes ventricular tachycardia in a human patient in need thereof, said method comprising administering to the patient effective amounts of one or more potassium channel blockers and one or more late sodium channel blockers.

In another aspect is provided, a method of preventing ventricular fibrillation in human patients susceptible to ventricular fibrillation, said method comprising administering to the patient effective amounts of one or more potassium channel blockers and one or more late sodium channel blockers.

In another aspect is provided, a method for modulating electrical and structural remodeling in a human patient in need thereof, said method comprising administering to the patient effective amounts of one or more potassium channel blockers and one or more late sodium channel blockers.

In another aspect is provided, a method of treating or preventing supraventricular tachyarrhythmia or ventricular tachyarrhythmia in a human patient in need thereof comprising administering to the patient effective amounts of one or more potassium channel blockers and one or more late sodium channel blockers.

In another aspect is provided, a method of preventing hospitalization and/or death in a human patient suffering from atrial fibrillation and/or atrial flutter comprising administering to the patient effective amounts of one or more potassium channel blockers and one or more late sodium channel blockers.

In another aspect is provided, a method of preventing stroke and/or congestive heart failure in a human patient in need thereof comprising administering to the patient effective amounts of one or more potassium channel blockers and one or more late sodium channel blockers.

In another aspect is provided, a pharmaceutical formulation comprising effective amounts of one or more late sodium channel blockers and one or more potassium channel blockers and a pharmaceutically acceptable carrier. The formulation may be formulated for intravenous administration or oral administration.

In one embodiment, the late sodium channel blocker is one or more compounds disclosed in US 2013/0012492; US 2013/0005706; US 2012/0289493; US 2009/0012103; US 2010/0197684; US 2009/0181986; US 2010/0113514; WO 2010/056865; US 2010/0125091; US 2010/0113449; US 2010/0113461; WO 2011/056985; US 2011/0021521; or WO 2012/003392, all of which are hereby incorporated by reference in its entirety.

In one embodiment, the potassium channel blocker is one or more compounds disclosed in U.S. Pat. Nos. 7,456,187; WO 2005/121149; 8,022,076; WO 2004/111057; WO 2007/066127; WO 2010/023445; WO 2010/023446; WO 2010/023448; or WO 2007/109211, all of which are hereby incorporated by reference in its entirety.

In one embodiment, the potassium channel blocker is a Kv1.5 potassium channel blocker or a IKACh potassium channel blocker. In another embodiment, the potassium channel blocker is one or more compounds disclosed in WO 2005/037780; US 2007/082037; US 2008/188509; WO 2005/041967; US 2009/203686; WO 2006/108837; WO 2009/079624; WO 2009/079630; WO 2010/023448; WO 2010/0139953; or WO 2010/0139967, all of which are hereby incorporated by reference.

### BRIEF DESCRIPTION OF THE FIGURES

FIG. 1 shows the synergistic effect of a late sodium channel blocker and a potassium channel blocker on the rabbit atrial effective refractory period.

### DETAILED DESCRIPTION

### 1. Definitions

As used in the present specification, the following words and phrases are generally intended to have the meanings as set forth below, except to the extent that the context in which they are used indicates otherwise.

It is to be noted that as used herein and in the claims, the singular forms “a,” “an” and “the” include plural referents unless the context clearly dictates otherwise. Thus, for example, reference to “a pharmaceutically acceptable carrier” in a composition includes two or more pharmaceutically acceptable carriers, and so forth.

“Comprising” is intended to mean that the compositions and methods include the recited elements, but do not exclude others. “Consisting essentially of” when used to define compositions and methods, shall mean excluding other elements of any essential significance to the combination for the intended use. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

The term “blocker” refers to an inhibitor, a modulator, etc.

The term “late sodium channel blocker” refers to a compound which inhibits, either selectively or nonselectively, the late sodium current (INaL). A compound's ability to inhibit late sodium current may be tested by the assay in the Examples. As stated above, compounds known to be late sodium channel blockers and therefore useful in the methods and formulations described herein are described in US 2013/0012492; US 2013/0005706; US 2012/0289493; US 2009/0012103; US 2010/0197684; US 2009/0181986; US 2010/0113514; WO 2010/056865; US 2010/0125091; US 2010/0113449; US 2010/0113461; WO 2011/056985; US 2011/0021521; or WO 2012/003392, all of which are hereby incorporated by reference in its entirety. In addition to the compounds being taught in the references just described, methods of making the compounds, dosage forms, dosage amounts, and the like are also described and this information is also incorporated by reference.

The term “potassium channel blocker” refers to a compound which inhibits the potassium channel. In some embodiments of the present disclosure, the “potassium channel blocker” is a Kv1.5 potassium channel blocker or a IKACh potassium channel blocker. In some embodiments, the potassium channel blocker does not substantially inhibit the I_{Kr} potassium channel.

In one embodiment, that potassium channel blocker interferes with a time-dependent potassium current. The current includes a hyperpolarization-activated time-dependent potassium current. The current is referred to as I_{KH}. This time-dependent K⁺ current, I_{KH}, has been observed upon hyperpolarization of PV and left atrial cardiomyocytes. In one embodiment, the compound interferes with an acetylcholine dependent potassium-carried current. In one embodiment, compound interferes with the inward-rectifying potassium channel (IKACh). This is more thoroughly described in US 2006/0094003. For example, and non-limitatively, interfering with an acetylcholine-dependent potassium-carried current includes inhibiting a repolarization current so as to increase a duration of a repolarization phase in cardiomyocytes. In some embodiments of the invention, the current is mediated by a Kir3 channel.

In some embodiments, the “potassium channel blocker” is a Kv1.5 potassium channel blocker. As used herein, a “Kv1.5 potassium channel blocker” is a compound which inhibits the Kv1.5 or Kv₍ᵤᵣ₎ potassium channels, which are known for the treatment of cardiac arrhythmia in the atria, such as atrial fibrillation.

In some embodiments, the “potassium channel blocker” is a IKACh potassium channel blocker. As used herein, a “IKACh potassium channel blocker” is a compound which inhibits the IKACh potassium channel. The inward-rectifing potassium channel (IKACh) is found in cardiac muscle (specifically, the sinoatrial node and atria). Inhibition of IKACh, which is a is a G protein-gated ion channel, is expected to maintain sinus rhythm in patients who have experienced episodes of atrial fibrillation.

As stated above, compounds known to be potassium channel blockers that are useful in the methods and pharmaceutical formulations described herein are described in U.S. Pat. No. 7,456,187; WO 2005/121149; 8,022,076; WO 2004/111057; WO 2007/066127; WO 2010/023445; WO 2010/023446; WO 2010/023448; WO 2007/109211, WO 2005/037780; US 2007/082037; US 2008/188509; WO 2005/041967; US 2009/203686; WO 2006/108837; WO 2009/079624; WO 2009/079630; WO 2010/023448; WO 2010/0139953; or WO 2010/0139967, all of which are hereby incorporated by reference. In addition to the compounds being taught in the references just described, methods of making the compounds, dosage forms, dosage amounts, and the like are also described and this information is also incorporated by reference. In one embodiment, the compound is XEN-D0103. A compound's ability to inhibit a potassium channel may be tested by the assays in the Examples.

The term “effective amount” refers to that amount of a compound, such as late sodium channel blocker or potassium channel blocker, that is sufficient to effect treatment, as defined below, when administered to a mammal in need of such treatment. The effective amount will vary depending upon the specific activity of the therapeutic agent being used, the severity of the patient's disease state, and the age, physical condition, existence of other disease states, and nutritional status of the patient. Additionally, other medication the patient may be receiving will effect the determination of the effective amount of the therapeutic agent to administer.

In one embodiment, the “effective amount” is a synergistic amount. “Synergistic” means that the effective amount of a potassium channel blocker when administered in combination with a late sodium channel blocker (or vice-versa) is greater than the predicted additive effective amounts of the potassium channel blocker and the late sodium channel blocker when administered alone. In one embodiment, the “effective amount” is less than the standard effective amount of one or both drugs when administered alone, meaning that the amount required for the desired effect when used in combination is lower than when the drug is used alone. In another embodiment, the effective amount is substantially the same as the standard effective amount of one or both drugs when administered alone.

The term “treatment” or “treating” means any treatment of a disease or condition in a subject, such as a mammal, including: 1) preventing or protecting against the disease or condition, that is, causing the clinical symptoms not to develop; 2) inhibiting the disease or condition, that is, arresting or suppressing the development of clinical symptoms; and/or 3) relieving the disease or condition that is, causing the regression of clinical symptoms. In some embodiments, the term “treatment” or “treating” refers to relieving the disease or condition that is, causing the regression of clinical symptoms.

As used herein, the term “preventing” refers to the prophylactic treatment of a patient in need thereof. The prophylactic treatment can be accomplished by providing an appropriate dose of a therapeutic agent to a subject at risk of suffering from an ailment, thereby substantially averting onset of the ailment.

It will be understood by those skilled in the art that in human medicine, it is not always possible to distinguish between “preventing” and “suppressing” since the ultimate inductive event or events may be unknown, latent, or the patient is not ascertained until well after the occurrence of the event or events. Therefore, as used herein the term “prophylaxis” is intended as an element of “treatment” to encompass both “preventing” and “suppressing” as defined herein. The term “protection,” as used herein, is meant to include “prophylaxis.”

The term “susceptible” refers to a patient who has had at least one occurrence of the indicated condition.

The term “patient” typically refers to a “mammal” which includes, without limitation, human, monkeys, rabbits, mice, domestic animals, such as dogs and cats, farm animals, such as cows, horses, or pigs, and laboratory animals. In some embodiments, the term patient refers to a human in need of treatment as defined herein.

“Atrial fibrillation” or “AF” occurs when the heart's two upper chambers (the right and left atria) quiver instead of beating and contracting rhythmically. Electrocardiographically, AF is characterized by a highly disorganized atrial electrical activity that often results in fast beating of the heart's two lower chambers (the right and left ventricles). Symptoms experienced by patients with AF include palpitation, fatigue, and dyspnea (shortness of breath).

There are three types of AF based on the presentation and duration of the arrhythmia: a) Paroxysmal AF: recurrent AF (>2 episodes) that starts and terminates spontaneously within 7 days (paroxysmal AF starts and stops spontaneously); b) Persistent AF: sustained AF that lasts longer than 7 days or requires termination by pharmacologic or electrical cardioversion (electrical shock); and c) Permanent AF: long standing AF (for >1 year duration) in which normal sinus rhythm cannot be maintained even after treatment, or when the patient and physician have decided to allow AF to continue without further efforts to restore sinus rhythm.

“Atrial flutter” is an abnormal heart rhythm that occurs in the atria of the heart. When it first occurs, it is usually associated with a fast heart rate or tachycardia (230-380 beats per minute (bpm)), and falls into the category of supra-ventricular tachycardias. While this rhythm occurs most often in individuals with cardiovascular disease (e.g. hypertension, coronary artery disease, and cardiomyopathy), it may occur spontaneously in people with otherwise normal hearts. It is typically not a stable rhythm, and frequently degenerates into atrial fibrillation (AF).

Both “electrical and structural remodeling” contribute to the pathogenesis of AF. Electrical triggers (after potentials) and arrhythmogenic substrate (re-entry) are two main causes for the initiation and maintenance of AF. “Electrical remodeling” is caused by malfunctioning of ion channels (mainly sodium, calcium, and potassium channels). “Structural remodeling” is caused by proliferation and differentiation of fibroblasts into myofibroblasts and enhanced connective tissue deposition. Structural remodeling results in the electrical dissociation between cardiac muscle bundles and heterogeneity in the electrical conduction in the atrium. Thus, inflammation and/or fibrosis of atrial tissue create a milieu conducive for AF. The electrical and structural remodeling of the atria leads to the perpetuation of AF. Hence, “AF begets AF”. Prolonged episodes of AF frequently cause mechanical dysfunction of the atrium resulting in adverse hemodynamic consequences and may contribute to heart failure.

“Ventricular fibrillation” occurs when the heart beats with rapid, erratic electrical impulses which causes pumping chambers in the heart (i.e. the ventricles) to quiver uselessly, rather than pump blood. Ventricular fibrillation requires immediate medical attention as blood pressure plummets, cutting off blood supply to vital organs. A person with ventricular fibrillation will collapse within seconds and soon will not be breathing or have a pulse. Symptoms include chest pain, rapid heartbeat (tachycardia), dizziness, nausea, shortness of breath, and loss of consciousness or fainting. It is not always known what causes ventricular fibrillation, but most cases of ventricular fibrillation begin as a rapid heartbeat called “ventricular tachycardia” or “VT”.

“Torsades de pointes (or TdP) ventricular tachycardia” refers to a specific variety of ventricular tachycardia that exhibits distinct characteristics on the electrocardiogram (ECG). The ECG reading in torsades demonstrates a rapid, polymorphic ventricular tachycardia with a characteristic twist of the QRS complex around the isoelectric baseline. It is also associated with a fall in arterial blood pressure, which can produce fainting. Although “torsades de pointes” is a rare ventricular arrhythmia, it can degenerate into “ventricular fibrillation”, which will lead to sudden death in the absence of medical intervention. Torsades de pointes is associated with long QT syndrome, a condition whereby prolonged QT intervals are visible on the ECG. Long QT intervals predispose the patient to an R-on-T phenomenon, where the R wave representing ventricular depolarization occurs simultaneously to the relative refractory period at the end of repolarization (represented by the latter half of the T-wave). An R-on-T can initiate torsades. Long QT syndrome can either be inherited as congenital mutations of ion channels carrying the cardiac impulse/action potential or acquired as a result of drugs that block these cardiac ion currents.

Common causes for torsades de pointes include diarrhea, hypomagnesemia, and hypokalemia. It is commonly seen in malnourished individuals and chronic alcoholics. Drug interactions such as erythromycin or moxifloxacin, taken concomitantly with inhibitors like nitroimidazole, dietary supplements, and various medications like methadone, lithium, tricyclic antidepressants or phenothiazines may also contribute. It can also be the side effect of some anti-arrhythmic medications such as sotalol, procainamide, and quinidine. Factors that are associated with an increased tendency toward torsades de pointes include: class IA antiarrhythmics, class III antiarrhythmics, hypomagnesemia, hypokalemia, hypocalcemia, hypoxia, acidosis, heart failure, left ventricular hypertrophy, slow heart rate, female gender, hypothermia, subarachnoid hemorrhage.

“AV conduction” or “atrioventricular conduction” is the forward conduction of the cardiac impulse from the atria to ventricles via the “atrioventricular node” or “AV node”, represented in an electrocardiogram by the P—R interval. The AV node is a part of electrical control system of the heart that electrically connects atrial and ventricular chambers and coordinates heart rate. The AV node is an area of specialized tissue between the atria and the ventricles of the heart, specifically in the posteroinferior region of the interatrial septum near the opening of the coronary sinus, which conducts the normal electrical impulse from the atria to the ventricles. “AV conduction” during normal cardiac rhythm occurs through two different pathways: the first has a slow conduction velocity but shorter refractory period, whereas the second has a faster conduction velocity but longer refractory period.

The term “modulate” means to increase or decrease or otherwise provide control.

“Modulating ventricular and/or atrial rate” has been shown to significantly improve AF. Typically, this has been accomplished with the use of a pacemaker, where the pacemaker detects the atrial beat and after a normal delay (0.1-0.2 seconds) triggers a ventricular beat, unless it has already happened—this can be achieved with a single pacing lead with electrodes in the right atrium (to sense) and ventricle (to sense and pace). The “atrial rate” is specific to the rate (measured in beats per unit time) of only the atrial beat. Pacemakers can also monitor and modulate the ventricular and/or atrial rhythm. The “ventricular and/or atrial rhythm” refers to the beat-to-beat time period of either the ventricular beat or the atrial beat.

Administering” or “administration” refers to the delivery of one or more therapeutic agents to a patient. In one embodiment, the administration is coadministration such that two or more therapeutic agents are delivered together at one time. In certain embodiments, two or more therapeutic agents can be coformulated into a single dosage form or “combined dosage unit”, or formulated separately and subsequently combined into a combined dosage unit, typically for intravenous administration or oral administration.

“Intravenous administration” is the administration of substances directly into a vein, or “intravenously”. Compared with other routes of administration, the intravenous (IV) route is the fastest way to deliver fluids and medications throughout the body. An infusion pump can allow precise control over the flow rate and total amount delivered, but in cases where a change in the flow rate would not have serious consequences, or if pumps are not available, the drip is often left to flow simply by placing the bag above the level of the patient and using the clamp to regulate the rate. Alternatively, a rapid infuser can be used if the patient requires a high flow rate and the IV access device is of a large enough diameter to accommodate it. This is either an inflatable cuff placed around the fluid bag to force the fluid into the patient or a similar electrical device that may also heat the fluid being infused. When a patient requires medications only at certain times, intermittent infusion is used, which does not require additional fluid. It can use the same techniques as an intravenous drip (pump or gravity drip), but after the complete dose of medication has been given, the tubing is disconnected from the IV access device. Some medications are also given by IV push or bolus, meaning that a syringe is connected to the IV access device and the medication is injected directly (slowly, if it might irritate the vein or cause a too-rapid effect). Once a medicine has been injected into the fluid stream of the IV tubing there must be some means of ensuring that it gets from the tubing to the patient. Usually this is accomplished by allowing the fluid stream to flow normally and thereby carry the medicine into the bloodstream; however, a second fluid injection is sometimes used, a “flush”, following the injection to push the medicine into the bloodstream more quickly.

“Oral administration” is a route of administration where a substance is taken through the mouth, and includes buccal, sublabial and sublingual administration, as well as enteral administration and that through the respiratory tract, unless made through e.g. tubing so the medication is not in direct contact with any of the oral mucosa. Typical form for the oral administration of therapeutic agents includes the use of tablets or capsules.

A “sustained release formulation” is a formulation which is designed to slowly release a therapeutic agent in the body over an extended period of time, whereas an “immediate release formulation” is an formulation which is designed to quickly release a therapeutic agent in the body over a shortened period of time. In some cases the immediate release formulation may be coated such that the therapeutic agent is only released once it reached the desired target in the body (e.g. the stomach).

Some of the more common “undesirable side effects of a potassium channel blocker” or “undesirable side effects of a late sodium channel blocker” include diarrhea, lack or loss of strength, abdominal or stomach pain, acid or sour stomach, belching, blistering, crusting, irritation, itching, or reddening of the skin, cracked, dry, or scaly skin, heartburn, indigestion, itching skin, nausea, rash, redness or discoloration of the skin, skin rash, encrusted, scaly, and oozing, skin rash, hives, itching, or redness, stomach discomfort, upset, or pain, swelling, and vomiting. Some of the less common or rare side effects include chest pain or discomfort, lightheadedness, dizziness, or fainting, shortness of breath, slow or irregular heartbeat, unusual tiredness, change in taste, increased sensitivity of the skin to sunlight, loss of taste and severe sunburn.

The term “alkyl” refers to a monoradical branched or unbranched saturated hydrocarbon chain having from 1 to 20 carbon atoms, or from 1 to 15 carbon atoms, or from 1 to 10 carbon atoms, or from 1 to 8 carbon atoms, or from 1 to 6 carbon atoms, or from 1 to 4 carbon atoms. This term is exemplified by groups such as methyl, ethyl, n-propyl, iso-propyl, n-butyl, iso-butyl, t-butyl, n-hexyl, n-decyl, tetradecyl, and the like.

The term “substituted alkyl” refers to:

1) an alkyl group as defined above, having 1, 2, 3, 4 or 5 substituents, (in some embodiments, 1, 2 or 3 substituents) selected from the group consisting of alkenyl, alkynyl, alkoxy, cycloalkyl, cycloalkenyl, cycloalkoxy, cycloalkenyloxy, acyl, acylamino, acyloxy, amino, substituted amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —S(O)-alkyl, —S(O)-cycloalkyl, —S(O)-heterocyclyl, —S(O)-aryl, —S(O)-heteroaryl, —S(O)₂-alkyl, —S(O)₂-cycloalkyl, —S(O)₂-heterocyclyl, —S(O)₂-aryl and —S(O)₂-heteroaryl. Unless otherwise constrained by the definition, all substituents may optionally be further substituted by 1, 2 or 3 substituents chosen from alkyl, alkenyl, alkynyl, carboxy, carboxyalkyl, aminocarbonyl, hydroxy, alkoxy, halogen, CF₃, amino, substituted amino, cyano, cycloalkyl, heterocyclyl, aryl, heteroaryl, and —S(O)ₙR^{a}, in which R^{a} is alkyl, aryl or heteroaryl and n is 0, 1 or 2; or

2) an alkyl group as defined above that is interrupted by 1-10 atoms (e.g. 1, 2, 3, 4 or 5 atoms) independently chosen from oxygen, sulfur and NR^{a}, where R^{a} is chosen from hydrogen, alkyl, cycloalkyl, alkenyl, cycloalkenyl, alkynyl, aryl, heteroaryl and heterocyclyl. All substituents may be optionally further substituted by alkyl, alkenyl, alkynyl, carboxy, carboxyalkyl, aminocarbonyl, hydroxy, alkoxy, halogen, CF₃, amino, substituted amino, cyano, cycloalkyl, heterocyclyl, aryl, heteroaryl, and —S(O)ₙR^{a}, in which R^{a} is alkyl, aryl or heteroaryl and n is 0, 1 or 2; or

3) an alkyl group as defined above that has both 1, 2, 3, 4 or 5 substituents as defined above and is also interrupted by 1-10 atoms (e.g. 1, 2, 3, 4 or 5 atoms) as defined above.

The term “lower alkyl” refers to a monoradical branched or unbranched saturated hydrocarbon chain having 1, 2, 3, 4, 5 or 6 carbon atoms. This term is exemplified by groups such as methyl, ethyl, n-propyl, iso-propyl, n-butyl, iso-butyl, t-butyl, n-hexyl, and the like.

The term “substituted lower alkyl” refers to lower alkyl as defined above having 1 to 5 substituents (in some embodiments, 1, 2 or 3 substituents), as defined for substituted alkyl or a lower alkyl group as defined above that is interrupted by 1, 2, 3, 4 or 5 atoms as defined for substituted alkyl or a lower alkyl group as defined above that has both 1, 2, 3, 4 or 5 substituents as defined above and is also interrupted by 1, 2, 3, 4 or 5 atoms as defined above.

The term “alkylene” refers to a diradical of a branched or unbranched saturated hydrocarbon chain, in some embodiments, having from 1 to 20 carbon atoms (e.g. 1-10 carbon atoms or 1, 2, 3, 4, 5 or 6 carbon atoms). This term is exemplified by groups such as methylene (—CH₂—), ethylene (—CH₂CH₂—), the propylene isomers (e.g., —CH₂CH₂CH₂— and —CH(CH₃)CH₂—), and the like.

The term “lower alkylene” refers to a diradical of a branched or unbranched saturated hydrocarbon chain, in some embodiments, having 1, 2, 3, 4, 5 or 6 carbon atoms.

The term “substituted alkylene” refers to an alkylene group as defined above having 1 to 5 substituents (in some embodiments, 1, 2 or 3 substituents) as defined for substituted alkyl.

The term “aralkyl” refers to an aryl group covalently linked to an alkylene group, where aryl and alkylene are defined herein. “Optionally substituted aralkyl” refers to an optionally substituted aryl group covalently linked to an optionally substituted alkylene group. Such aralkyl groups are exemplified by benzyl, phenylethyl, 3-(4-methoxyphenyl)propyl, and the like.

The term “aralkyloxy” refers to the group —O-aralkyl. “Optionally substituted aralkyloxy” refers to an optionally substituted aralkyl group covalently linked to an optionally substituted alkylene group. Such aralkyl groups are exemplified by benzyloxy, phenylethyloxy, and the like.

The term “alkenyl” refers to a monoradical of a branched or unbranched unsaturated hydrocarbon group having from 2 to 20 carbon atoms (in some embodiments, from 2 to 10 carbon atoms, e.g. 2 to 6 carbon atoms) and having from 1 to 6 carbon-carbon double bonds, e.g. 1, 2 or 3 carbon-carbon double bonds. In some embodiments, alkenyl groups include ethenyl (or vinyl, i.e. —CH═CH₂), 1-propylene (or allyl, i.e. —CH₂CH═CH₂), isopropylene (—C(CH₃)═CH₂), and the like.

The term “lower alkenyl” refers to alkenyl as defined above having from 2 to 6 carbon atoms.

The term “substituted alkenyl” refers to an alkenyl group as defined above having 1 to 5 substituents (in some embodiments, 1, 2 or 3 substituents) as defined for substituted alkyl.

The term “alkenylene” refers to a diradical of a branched or unbranched unsaturated hydrocarbon group having from 2 to 20 carbon atoms (in some embodiments, from 2 to 10 carbon atoms, e.g. 2 to 6 carbon atoms) and having from 1 to 6 carbon-carbon double bonds, e.g. 1, 2 or 3 carbon-carbon double bonds.

The term “alkynyl” refers to a monoradical of an unsaturated hydrocarbon, in some embodiments, having from 2 to 20 carbon atoms (in some embodiments, from 2 to 10 carbon atoms, e.g. 2 to 6 carbon atoms) and having from 1 to 6 carbon-carbon triple bonds e.g. 1, 2 or 3 carbon-carbon triple bonds. In some embodiments, alkynyl groups include ethynyl (—C≡CH), propargyl (or propynyl, i.e. —C≡CCH₃), and the like.

The term “substituted alkynyl” refers to an alkynyl group as defined above having 1 to 5 substituents (in some embodiments, 1, 2 or 3 substituents) as defined for substituted alkyl.

The term “alkynylene” refers to a diradical of an unsaturated hydrocarbon, in some embodiments, having from 2 to 20 carbon atoms (in some embodiments, from 2 to 10 carbon atoms, e.g. 2 to 6 carbon atoms) and having from 1 to 6 carbon-carbon triple bonds e.g. 1, 2 or 3 carbon-carbon triple bonds.

The term “hydroxy” or “hydroxyl” refers to a group —OH.

The term “alkoxy” refers to the group R—O—, where R is alkyl or —Y—Z, in which Y is alkylene and Z is alkenyl or alkynyl, where alkyl, alkenyl and alkynyl are as defined herein. In some embodiments, alkoxy groups are alkyl-O— and includes, by way of example, methoxy, ethoxy, n-propoxy, iso-propoxy, n-butoxy, tert-butoxy, sec-butoxy, n-pentoxy, n-hexyloxy, 1,2-dimethylbutoxy, and the like.

The term “lower alkoxy” refers to the group R—O— in which R is optionally substituted lower alkyl. This term is exemplified by groups such as methoxy, ethoxy, n-propoxy, iso-propoxy, n-butoxy, iso-butoxy, t-butoxy, n-hexyloxy, and the like.

The term “substituted alkoxy” refers to the group R—O—, where R is substituted alkyl or —Y—Z, in which Y is substituted alkylene and Z is substituted alkenyl or substituted alkynyl, where substituted alkyl, substituted alkenyl and substituted alkynyl are as defined herein.

The term “C₁₋₃ haloalkyl” refers to an alkyl group having from 1 to 3 carbon atoms covalently bonded to from 1 to 7, or from 1 to 6, or from 1 to 3, halogen(s), where alkyl and halogen are defined herein. In some embodiments, C₁₋₃ haloalkyl includes, by way of example, trifluoromethyl, difluoromethyl, fluoromethyl, 2,2,2-trifluoroethyl, 2,2-difluoroethyl, 2-fluoroethyl, 3,3,3-trifluoropropyl, 3,3-difluoropropyl, 3-fluoropropyl.

The term “cycloalkyl” refers to cyclic alkyl groups of from 3 to 20 carbon atoms, or from 3 to 10 carbon atoms, having a single cyclic ring or multiple condensed rings. Such cycloalkyl groups include, by way of example, single ring structures such as cyclopropyl, cyclobutyl, cyclopentyl, cyclooctyl and the like or multiple ring structures such as adamantanyl and bicyclo[2.2.1]heptanyl or cyclic alkyl groups to which is fused an aryl group, for example indanyl, and the like, provided that the point of attachment is through the cyclic alkyl group.

The term “cycloalkenyl” refers to cyclic alkyl groups of from 3 to 20 carbon atoms having a single cyclic ring or multiple condensed rings and having at least one double bond and in some embodiments, from 1 to 2 double bonds.

The terms “substituted cycloalkyl” and “substituted cycloalkenyl” refer to cycloalkyl or cycloalkenyl groups having 1, 2, 3, 4 or 5 substituents (in some embodiments, 1, 2 or 3 substituents), selected from the group consisting of alkyl, alkenyl, alkynyl, alkoxy, cycloalkyl, cycloalkenyl, cycloalkoxy, cycloalkenyloxy, acyl, acylamino, acyloxy, amino, substituted amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —S(O)-alkyl, —S(O)-cycloalkyl, —S(O)-heterocyclyl, —S(O)-aryl, —S(O)-heteroaryl, —S(O)₂-alkyl, —S(O)₂-cycloalkyl, —S(O)₂-heterocyclyl, —S(O)₂-aryl and —S(O)₂-heteroaryl. The term “substituted cycloalkyl” also includes cycloalkyl groups wherein one or more of the annular carbon atoms of the cycloalkyl group has an oxo group bonded thereto. In addition, a substituent on the cycloalkyl or cycloalkenyl may be attached to the same carbon atom as, or is geminal to, the attachment of the substituted cycloalkyl or cycloalkenyl to the 6,7-ring system. Unless otherwise constrained by the definition, all substituents may optionally be further substituted by 1, 2 or 3 substituents chosen from alkyl, alkenyl, alkynyl, carboxy, carboxyalkyl, aminocarbonyl, hydroxy, alkoxy, halogen, CF₃, amino, substituted amino, cyano, cycloalkyl, heterocyclyl, aryl, heteroaryl, and —S(O)ₙR^{a}, in which R^{a} is alkyl, aryl or heteroaryl and n is 0, 1 or 2.

The term “cycloalkoxy” refers to the group cycloalkyl-O—.

The term “substituted cycloalkoxy” refers to the group substituted cycloalkyl-O—.

The term “cycloalkenyloxy” refers to the group cycloalkenyl-O—.

The term “substituted cycloalkenyloxy” refers to the group substituted cycloalkenyl-O—.

The term “aryl” refers to an aromatic carbocyclic group of 6 to 20 carbon atoms having a single ring (e.g., phenyl) or multiple rings (e.g., biphenyl) or multiple condensed (fused) rings (e.g., naphthyl, fluorenyl and anthryl). In some embodiments, aryls include phenyl, fluorenyl, naphthyl, anthryl, and the like.

Unless otherwise constrained by the definition for the aryl substituent, such aryl groups can optionally be substituted with 1, 2, 3, 4 or 5 substituents (in some embodiments, 1, 2 or 3 substituents), selected from the group consisting of alkyl, alkenyl, alkynyl, alkoxy, cycloalkyl, cycloalkenyl, cycloalkoxy, cycloalkenyloxy, acyl, acylamino, acyloxy, amino, substituted amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —S(O)-alkyl, —S(O)-cycloalkyl, —S(O)-heterocyclyl, —S(O)-aryl, —S(O)-heteroaryl, —S(O)₂-alkyl, —S(O)₂-cycloalkyl, —S(O)₂-heterocyclyl, —S(O)₂-aryl and —S(O)₂-heteroaryl. Unless otherwise constrained by the definition, all substituents may optionally be further substituted by 1, 2 or 3 substituents chosen from alkyl, alkenyl, alkynyl, carboxy, carboxyalkyl, aminocarbonyl, hydroxy, alkoxy, halogen, CF₃, amino, substituted amino, cyano, cycloalkyl, heterocyclyl, aryl, heteroaryl, and —S(O)ₙR^{a}, in which R^{a} is alkyl, aryl or heteroaryl and n is 0, 1 or 2.

The term “aryloxy” refers to the group aryl-O— wherein the aryl group is as defined above, and includes optionally substituted aryl groups as also defined above. The term “arylthio” refers to the group R—S—, where R is as defined for aryl.

The term “heterocyclyl,” “heterocycle,” or “heterocyclic” refers to a monoradical saturated group having a single ring or multiple condensed rings, having from 1 to 40 carbon atoms and from 1 to 10 hetero atoms, and from 1 to 4 heteroatoms, selected from nitrogen, sulfur, phosphorus, and/or oxygen within the ring. In some embodiments, the heterocyclyl,” “heterocycle,” or “heterocyclic” group is linked to the remainder of the molecule through one of the heteroatoms within the ring.

Unless otherwise constrained by the definition for the heterocyclic substituent, such heterocyclic groups can be optionally substituted with 1 to 5 substituents (in some embodiments, 1, 2 or 3 substituents), selected from the group consisting of alkyl, alkenyl, alkynyl, alkoxy, cycloalkyl, cycloalkenyl, cycloalkoxy, cycloalkenyloxy, acyl, acylamino, acyloxy, amino, substituted amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —S(O)-alkyl, —S(O)-cycloalkyl, —S(O)-heterocyclyl, —S(O)-aryl, —S(O)-heteroaryl, —S(O)₂-alkyl, —S(O)₂-cycloalkyl, —S(O)₂-heterocyclyl, —S(O)₂-aryl and —S(O)₂-heteroaryl. In addition, a substituent on the heterocyclic group may be attached to the same carbon atom as, or is geminal to, the attachment of the substituted heterocyclic group to the 6,7-ring system. Unless otherwise constrained by the definition, all substituents may optionally be further substituted by 1, 2 or 3 substituents chosen from alkyl, alkenyl, alkynyl, carboxy, carboxyalkyl, aminocarbonyl, hydroxy, alkoxy, halogen, CF₃, amino, substituted amino, cyano, cycloalkyl, heterocyclyl, aryl, heteroaryl, and —S(O)ₙR^{a}, in which R^{a} is alkyl, aryl or heteroaryl and n is 0, 1 or 2. Examples of heterocyclics include tetrahydrofuranyl, morpholino, piperidinyl, and the like.

The term “heterocyclooxy” refers to the group —O-heterocyclyl.

The term “heteroaryl” refers to a group comprising single or multiple rings comprising 1 to 15 carbon atoms and 1 to 4 heteroatoms selected from oxygen, nitrogen and sulfur within at least one ring. The term “heteroaryl” is generic to the terms “aromatic heteroaryl” and “partially saturated heteroaryl”. The term “aromatic heteroaryl” refers to a heteroaryl in which at least one ring is aromatic, regardless of the point of attachment. Examples of aromatic heteroaryls include pyrrole, thiophene, pyridine, quinoline, pteridine.

The term “partially saturated heteroaryl” refers to a heteroaryl having a structure equivalent to an underlying aromatic heteroaryl which has had one or more double bonds in an aromatic ring of the underlying aromatic heteroaryl saturated. Examples of partially saturated heteroaryls include dihydropyrrole, dihydropyridine, chroman, 2-oxo-1,2-dihydropyridin-4-yl, and the like.

Unless otherwise constrained by the definition for the heteroaryl substituent, such heteroaryl groups can be optionally substituted with 1 to 5 substituents (in some embodiments, 1, 2 or 3 substituents) selected from the group consisting alkyl, alkenyl, alkynyl, alkoxy, cycloalkyl, cycloalkenyl, cycloalkoxy, cycloalkenyloxy, acyl, acylamino, acyloxy, amino, substituted amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —S(O)-alkyl, —S(O)-cycloalkyl, —S(O)-heterocyclyl, —S(O)-aryl, —S(O)-heteroaryl, —S(O)₂-alkyl, —S(O)₂-cycloalkyl, —S(O)₂-heterocyclyl, —S(O)₂-aryl and —S(O)₂-heteroaryl. Unless otherwise constrained by the definition, all substituents may optionally be further substituted by 1, 2 or 3 substituents chosen from alkyl, alkenyl, alkynyl, carboxy, carboxyalkyl, aminocarbonyl, hydroxy, alkoxy, halogen, CF₃, amino, substituted amino, cyano, cycloalkyl, heterocyclyl, aryl, heteroaryl, and —S(O)ₙR^{a}, in which R^{a} is alkyl, aryl or heteroaryl and n is 0, 1 or 2. Such heteroaryl groups can have a single ring (e.g., pyridyl or furyl) or multiple condensed rings (e.g., indolizinyl, benzothiazole or benzothienyl). Examples of nitrogen heterocyclyls and heteroaryls include, but are not limited to, pyrrole, imidazole, pyrazole, pyridine, pyrazine, pyrimidine, pyridazine, indolizine, isoindole, indole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, and the like as well as N-alkoxy-nitrogen containing heteroaryl compounds.

The term “heteroaryloxy” refers to the group heteroaryl-O—.

The term “amino” refers to the group —NH₂.

The term “substituted amino” refers to the group —NRR where each R is independently selected from the group consisting of hydrogen, alkyl, cycloalkyl, aryl, heteroaryl and heterocyclyl provided that both R groups are not hydrogen or a group —Y—Z, in which Y is optionally substituted alkylene and Z is alkenyl, cycloalkenyl or alkynyl. Unless otherwise constrained by the definition, all substituents may optionally be further substituted by 1, 2 or 3 substituents chosen from alkyl, alkenyl, alkynyl, carboxy, carboxyalkyl, aminocarbonyl, hydroxy, alkoxy, halogen, CF₃, amino, substituted amino, cyano, cycloalkyl, heterocyclyl, aryl, heteroaryl, and —S(O)ₙR^{a}, in which R^{a} is alkyl, aryl or heteroaryl and n is 0, 1 or 2.

The term “alkyl amine” refers to R—NH₂ in which R is optionally substituted alkyl.

The term “dialkyl amine” refers to R—NHR in which each R is independently an optionally substituted alkyl.

The term “trialkyl amine” refers to NR₃ in which each R is independently an optionally substituted alkyl.

The term “cyano” refers to the group —CN.

The term “azido” refers to a group [symbol] .

The term “keto” or “oxo” refers to a group ═O.

The term “carboxy” refers to a group —C(O)—OH.

The term “ester” or “carboxyester” refers to the group —C(O)OR, where R is alkyl, cycloalkyl, aryl, heteroaryl or heterocyclyl, which may be optionally further substituted by alkyl, alkoxy, halogen, CF₃, amino, substituted amino, cyano or —S(O)ₙR^{a}, in which R^{a} is alkyl, aryl or heteroaryl and n is 0, 1 or 2.

The term “acyl” denotes the group —C(O)R, in which R is hydrogen, alkyl, cycloalkyl, heterocyclyl, aryl or heteroaryl. Unless otherwise constrained by the definition, all substituents may optionally be further substituted by 1, 2 or 3 substituents selected from the group consisting of alkyl, alkenyl, alkynyl, carboxy, carboxyalkyl, aminocarbonyl, hydroxy, alkoxy, halogen, CF₃, amino, substituted amino, cyano, cycloalkyl, heterocyclyl, aryl, heteroaryl, and —S(O)ₙR^{a}, in which R^{a} is alkyl, aryl or heteroaryl and n is 0, 1 or 2.

The term “carboxyalkyl” refers to the groups —C(O)O-alkyl or —C(O)O-cycloalkyl, where alkyl and cycloalkyl are as defined herein, and may be optionally further substituted by alkyl, alkenyl, alkynyl, carboxy, carboxyalkyl, aminocarbonyl, hydroxy, alkoxy, halogen, CF₃, amino, substituted amino, cyano, cycloalkyl, heterocyclyl, aryl, heteroaryl, and —S(O)ₙR^{a}, in which R^{a} is alkyl, aryl or heteroaryl and n is 0, 1 or 2.

The term “aminocarbonyl” refers to the group —C(O)NRR where each R is independently hydrogen, alkyl, cycloalkyl, aryl, heteroaryl, or heterocyclyl, or where both R groups are joined to form a heterocyclic group (e.g., morpholino). Unless otherwise constrained by the definition, all substituents may optionally be further substituted by 1, 2 or 3 substituents selected from the group consisting of alkyl, alkenyl, alkynyl, carboxy, carboxyalkyl, aminocarbonyl, hydroxy, alkoxy, halogen, CF₃, amino, substituted amino, cyano, cycloalkyl, heterocyclyl, aryl, heteroaryl, and —S(O)ₙR^{a}, in which R^{a} is alkyl, aryl or heteroaryl and n is 0, 1 or 2.

The term “acyloxy” refers to the group —OC(O)—R, in which R is alkyl, cycloalkyl, heterocyclyl, aryl or heteroaryl. Unless otherwise constrained by the definition, all substituents may optionally be further substituted by 1, 2 or 3 substituents selected from the group consisting of alkyl, alkenyl, alkynyl, carboxy, carboxyalkyl, aminocarbonyl, hydroxy, alkoxy, halogen, CF₃, amino, substituted amino, cyano, cycloalkyl, heterocyclyl, aryl, heteroaryl, and —S(O)ₙR^{a}, in which R^{a} is alkyl, aryl or heteroaryl and n is 0, 1 or 2.

The term “acylamino” refers to the group —NRC(O)R where each R is independently hydrogen, alkyl, cycloalkyl, aryl, heteroaryl or heterocyclyl. Unless otherwise constrained by the definition, all substituents may optionally be further substituted by 1, 2 or 3 substituents selected from the group consisting of alkyl, alkenyl, alkynyl, carboxy, carboxyalkyl, aminocarbonyl, hydroxy, alkoxy, halogen, CF₃, amino, substituted amino, cyano, cycloalkyl, heterocyclyl, aryl, heteroaryl, and —S(O)ₙR^{a}, in which R^{a} is alkyl, aryl or heteroaryl and n is 0, 1 or 2.

The term “alkoxycarbonylamino” refers to the group —N(R^{d})C(O)OR in which R is alkyl and R^{d} is hydrogen or alkyl. Unless otherwise constrained by the definition, each alkyl may optionally be further substituted by 1, 2 or 3 substituents selected from the group consisting of alkyl, alkenyl, alkynyl, carboxy, carboxyalkyl, aminocarbonyl, hydroxy, alkoxy, halogen, CF₃, amino, substituted amino, cyano, cycloalkyl, heterocyclyl, aryl, heteroaryl, and —S(O)ₙR^{a}, in which R^{a} is alkyl, aryl or heteroaryl and n is 0, 1 or 2.

The term “aminocarbonylamino” refers to the group —NR^{c}C(O)NRR, wherein R^{c} is hydrogen or alkyl and each R is hydrogen, alkyl, cycloalkyl, aryl, heteroaryl or heterocyclyl. Unless otherwise constrained by the definition, all substituents may optionally be further substituted by 1, 2 or 3 substituents selected from the group consisting of alkyl, alkenyl, alkynyl, carboxy, carboxyalkyl, aminocarbonyl, hydroxy, alkoxy, halogen, CF₃, amino, substituted amino, cyano, cycloalkyl, heterocyclyl, aryl, heteroaryl, and —S(O)ₙR^{a}, in which R^{a} is alkyl, aryl or heteroaryl and n is 0, 1 or 2.

The term “thiol” refers to the group —SH.

The term “thiocarbonyl” refers to a group ═S.

The term “alkylthio” refers to the group —S-alkyl.

The term “substituted alkylthio” refers to the group —S-substituted alkyl.

The term “heterocyclylthio” refers to the group —S-heterocyclyl.

The term “arylthio” refers to the group —S-aryl.

The term “heteroarylthiol” refers to the group —S-heteroaryl wherein the heteroaryl group is as defined above including optionally substituted heteroaryl groups as also defined above.

The term “sulfoxide” refers to a group —S(O)R, in which R is alkyl, cycloalkyl, heterocyclyl, aryl or heteroaryl. “Substituted sulfoxide” refers to a group —S(O)R, in which R is substituted alkyl, substituted cycloalkyl, substituted heterocyclyl, substituted aryl or substituted heteroaryl, as defined herein.

The term “sulfone” refers to a group —S(O)₂R, in which R is alkyl, cycloalkyl, heterocyclyl, aryl or heteroaryl. “Substituted sulfone” refers to a group —S(O)₂R, in which R is substituted alkyl, substituted cycloalkyl, substituted heterocyclyl, substituted aryl or substituted heteroaryl, as defined herein.

The term “aminosulfonyl” refers to the group —S(O)₂NRR, wherein each R is independently hydrogen, alkyl, cycloalkyl, aryl, heteroaryl or heterocyclyl. Unless otherwise constrained by the definition, all substituents may optionally be further substituted by 1, 2 or 3 substituents selected from the group consisting of alkyl, alkenyl, alkynyl, carboxy, carboxyalkyl, aminocarbonyl, hydroxy, alkoxy, halogen, CF₃, amino, substituted amino, cyano, cycloalkyl, heterocyclyl, aryl, heteroaryl, and —S(O)ₙR^{a}, in which R^{a} is alkyl, aryl or heteroaryl and n is 0, 1 or 2.

The term “hydroxyamino” refers to the group —NHOH.

The term “alkoxyamino” refers to the group —NHOR in which R is optionally substituted alkyl.

The term “halogen” or “halo” refers to fluoro, bromo, chloro and iodo.

“Optional” or “optionally” means that the subsequently described event or circumstance may or may not occur, and that the description includes instances where said event or circumstance occurs and instances in which it does not.

A “substituted” group includes embodiments in which a monoradical substituent is bound to a single atom of the substituted group (e.g. forming a branch), and also includes embodiments in which the substituent may be a diradical bridging group bound to two adjacent atoms of the substituted group, thereby forming a fused ring on the substituted group.

Where a given group (moiety) is described herein as being attached to a second group and the site of attachment is not explicit, the given group may be attached at any available site of the given group to any available site of the second group. For example, a “lower alkyl-substituted phenyl”, where the attachment sites are not explicit, may have any available site of the lower alkyl group attached to any available site of the phenyl group. In this regard, an “available site” is a site of the group at which a hydrogen of the group may be replaced with a substituent.

It is understood that in all substituted groups defined above, polymers arrived at by defining substituents with further substituents to themselves (e.g., substituted aryl having a substituted aryl group as a substituent which is itself substituted with a substituted aryl group, etc.) are not intended for inclusion herein. Also not included are infinite numbers of substituents, whether the substituents are the same or different. In such cases, the maximum number of such substituents is three. Each of the above definitions is thus constrained by a limitation that, for example, substituted aryl groups are limited to substituted aryl-(substituted aryl)-substituted aryl.

The term “compound” is intended to encompass the compounds of the disclosure, and the pharmaceutically acceptable salts, pharmaceutically acceptable esters, hydrates, polymorphs, and prodrugs of such compounds. Additionally, the compounds of the disclosure may possess one or more asymmetric centers, and can be produced as a racemic mixture or as individual enantiomers or diastereoisomers. The number of stereoisomers present in any given compound depends upon the number of asymmetric centers present (there are 2ⁿ stereoisomers possible where n is the number of asymmetric centers). The individual stereoisomers may be obtained by resolving a racemic or non-racemic mixture of an intermediate at some appropriate stage of the synthesis, or by resolution of the compound by conventional means. The individual stereoisomers (including individual enantiomers and diastereoisomers) as well as racemic and non-racemic mixtures of stereoisomers are encompassed within the scope of the present disclosure, all of which are intended to be depicted by the structures of this specification unless otherwise specifically indicated.

“Isomers” are different compounds that have the same molecular formula. Isomers include stereoisomers, enantiomers, and diastereomers.

“Stereoisomers” are isomers that differ only in the way the atoms are arranged in space.

“Enantiomers” are a pair of stereoisomers that are non-superimposable mirror images of each other. A 1:1 mixture of a pair of enantiomers is a “racemic” mixture. The term “(±)” is used to designate a racemic mixture where appropriate.

“Diastereoisomers” are stereoisomers that have at least two asymmetric atoms, but which are not mirror-images of each other.

The absolute stereochemistry is specified according to the Cahn Ingold Prelog R S system. When the compound is a pure enantiomer the stereochemistry at each chiral carbon may be specified by either R or S. Resolved compounds whose absolute configuration is unknown are designated (+) or (−) depending on the direction (dextro- or laevorotary) that they rotate the plane of polarized light at the wavelength of the sodium D line.

Some of the compounds exist as tautomeric isomers. Tautomeric isomers are in equilibrium with one another. For example, amide containing compounds may exist in equilibrium with imidic acid tautomers. Regardless of which tautomer is shown, and regardless of the nature of the equilibrium among tautomers, the compounds are understood by one of ordinary skill in the art to comprise both amide and imidic acid tautomers. Non-limiting examples of these tautomers are shown below:

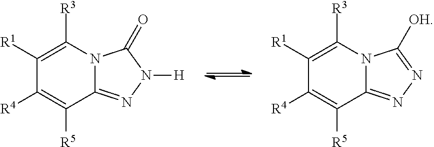

The term “polymorph” refers to different crystal structures of a crystalline compound. The different polymorphs may result from differences in crystal packing (packing polymorphism) or differences in packing between different conformers of the same molecule (conformational polymorphism).

The term “solvate” refers to a complex formed by the combining of a compound of Formula I or II and a solvent.

The term “hydrate” refers to the complex formed by the combining of a compound of Formula I or II and water.

The term “prodrug” refers to compounds of Formula I or II that include chemical groups which, in vivo, can be converted and/or can be split off from the remainder of the molecule to provide for the active drug, a pharmaceutically acceptable salt thereof, or a biologically active metabolite thereof.

The term “compound” is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the invention include isotopes of hydrogen, carbon, nitrogen, oxygen, phosphorous, fluorine, and chlorine, such as, but not limited to ²H (deuterium, D), ³H (tritium), ¹¹C, ¹³C, ¹⁴C, ¹⁵N, ¹⁸F, ³¹P, ³²P, ³⁵S, ³⁶Cl and ¹²⁵I. Various isotopically labeled compounds of the present invention, for example those into which radioactive isotopes such as ³H, ¹³C, and ¹⁴C are incorporated. Such isotopically labeled compounds may be useful in metabolic studies, reaction kinetic studies, detection or imaging techniques, such as positron emission tomography (PET) or single-photon emission computed tomography (SPECT) including drug or substrate tissue distribution assays, or in radioactive treatment of patients.

Any formula or structure given herein, including compounds of Formula I or II, is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the disclosure include isotopes of hydrogen, carbon, nitrogen, oxygen, phosphorous, fluorine and chlorine, such as, but not limited to ²H (deuterium, D), ³H (tritium), ¹¹C, ¹³C, ¹⁴C, ¹⁵N, ¹⁸F, ³¹P,³²P, ³⁵S, ³⁶Cl and ¹²⁵I. Various isotopically labeled compounds of the present disclosure, for example those into which radioactive isotopes such as ³H, ¹³C and ¹⁴C are incorporated. Such isotopically labelled compounds may be useful in metabolic studies, reaction kinetic studies, detection or imaging techniques, such as positron emission tomography (PET) or single-photon emission computed tomography (SPECT) including drug or substrate tissue distribution assays or in radioactive treatment of patients.

The disclosure also includes compounds of Formula I or II in which from 1 to n hydrogens attached to a carbon atom is/are replaced by deuterium, in which n is the number of hydrogens in the molecule. Such compounds exhibit increased resistance to metabolism and are thus useful for increasing the half life of any compound of Formula I or II when administered to a mammal. See, for example, Foster, “Deuterium Isotope Effects in Studies of Drug Metabolism”, Trends Pharmacol. Sci. 5(12):524-527 (1984). Such compounds are synthesized by means well known in the art, for example by employing starting materials in which one or more hydrogens have been replaced by deuterium.

Deuterium labelled or substituted therapeutic compounds of the disclosure may have improved DMPK (drug metabolism and pharmacokinetics) properties, relating to distribution, metabolism and excretion (ADME). Substitution with heavier isotopes such as deuterium may afford certain therapeutic advantages resulting from greater metabolic stability, for example increased in vivo half-life, reduced dosage requirements and/or an improvement in therapeutic index. An ¹⁸F labeled compound may be useful for PET or SPECT studies. Isotopically labeled compounds of this disclosure and prodrugs thereof can generally be prepared by carrying out the procedures disclosed in the schemes or in the examples and preparations described below by substituting a readily available isotopically labeled reagent for a non-isotopically labeled reagent. It is understood that deuterium in this context is regarded as a substituent in the compound of Formula I or II.

The concentration of such a heavier isotope, specifically deuterium, may be defined by an isotopic enrichment factor. In the compounds of this disclosure any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition. Accordingly, in the compounds of this disclosure any atom specifically designated as a deuterium (D) is meant to represent deuterium.

In many cases, the compounds of this disclosure are capable of forming acid and/or base salts by virtue of the presence of amino and/or carboxyl groups or groups similar thereto.

The term “pharmaceutically acceptable salt” of a given compound refers to salts that retain the biological effectiveness and properties of the given compound, and which are not biologically or otherwise undesirable. Pharmaceutically acceptable base addition salts can be prepared from inorganic and organic bases. Salts derived from inorganic bases include, by way of example only, sodium, potassium, lithium, ammonium, calcium and magnesium salts. Salts derived from organic bases include, but are not limited to, salts of primary, secondary and tertiary amines, such as alkyl amines, dialkyl amines, trialkyl amines, substituted alkyl amines, di(substituted alkyl) amines, tri(substituted alkyl) amines, alkenyl amines, dialkenyl amines, trialkenyl amines, substituted alkenyl amines, di(substituted alkenyl) amines, tri(substituted alkenyl) amines, cycloalkyl amines, di(cycloalkyl) amines, tri(cycloalkyl) amines, substituted cycloalkyl amines, disubstituted cycloalkyl amine, trisubstituted cycloalkyl amines, cycloalkenyl amines, di(cycloalkenyl) amines, tri(cycloalkenyl) amines, substituted cycloalkenyl amines, disubstituted cycloalkenyl amine, trisubstituted cycloalkenyl amines, aryl amines, diaryl amines, triaryl amines, heteroaryl amines, diheteroaryl amines, triheteroaryl amines, heterocyclic amines, diheterocyclic amines, triheterocyclic amines, mixed di- and tri-amines where at least two of the substituents on the amine are different and are selected from the group consisting of alkyl, substituted alkyl, alkenyl, substituted alkenyl, cycloalkyl, substituted cycloalkyl, cycloalkenyl, substituted cycloalkenyl, aryl, heteroaryl, heterocyclic, and the like. Also included are amines where the two or three substituents, together with the amino nitrogen, form a heterocyclic or heteroaryl group. Amines are of general structure N(R³⁰)(R³¹)(R³²), wherein mono-substituted amines have 2 of the three substituents on nitrogen (R³⁰, R³¹, and R³²) as hydrogen, di-substituted amines have 1 of the three substituents on nitrogen (R³⁰, R³¹, and R³²) as hydrogen, whereas tri-substituted amines have none of the three substituents on nitrogen (R³⁰, R³¹, and R³²) as hydrogen. R³⁰, R³¹, and R³² are selected from a variety of substituents such as hydrogen, optionally substituted alkyl, aryl, heteroaryl, cycloalkyl, cycloalkenyl, heterocyclyl, and the like. The above-mentioned amines refer to the compounds wherein either one, two, or three substituents on the nitrogen are as listed in the name. For example, the term “cycloalkenyl amine” refers to cycloalkenyl-NH₂, wherein “cycloalkenyl” is as defined herein. The term “diheteroarylamine” refers to NH(heteroaryl)₂, wherein “heteroaryl” is as defined herein, and so on.

Specific examples of suitable amines include, by way of example only, isopropylamine, trimethyl amine, diethyl amine, tri(iso-propyl) amine, tri(n-propyl) amine, ethanolamine, 2-dimethylaminoethanol, tromethamine, lysine, arginine, histidine, caffeine, procaine, hydrabamine, choline, betaine, ethylenediamine, glucosamine, N-alkylglucamines, theobromine, purines, piperazine, piperidine, morpholine, N-ethylpiperidine, and the like.

Pharmaceutically acceptable acid addition salts may be prepared from inorganic and organic acids. Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like.

In certain embodiments, it is contemplated that the late sodium channel blocker and/or potassium channel blocker as used herein has not been sufficiently ionized and may be in the form of a co-crystal. In one embodiment, the present invention provides a co-crystal composition comprising a co-crystal of late sodium channel blocker and/or potassium channel blocker, wherein said co-crystal comprises late sodium channel blocker and/or potassium channel blocker and a co-crystal former. The term “co-crystal” refers a crystalline material which comprises late sodium channel blocker and/or potassium channel blocker and one or more co-crystal formers, such as a pharmaceutically acceptable salt. In certain embodiments, the co-crystal can have an improved property as compared to the free form (i.e., the free molecule, zwitter ion, hydrate, solvate, etc.) or a salt (which includes salt hydrates and solvates). In further embodiments, the improved property is selected from the group consisting of: increased solubility, increased dissolution, increased bioavailability, increased dose response, decreased hygroscopicity, a crystalline form of a normally amorphous compound, a crystalline form of a difficult to salt or unsaltable compound, decreased form diversity, more desired morphology, and the like. Methods for making and characterizing co-crystals are well known to those of skill in the art.

As used herein, “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, or unless otherwise indicated herein, its use in the therapeutic compositions is contemplated. Supplementary active ingredients can also be incorporated into the compositions.

### 2. Methods

Generally, the present disclosure relates to methods of treating or preventing atrial fibrillation and/or atrial flutter. The method comprises administration of an effective amount of a potassium channel blocker and an effective amount of a late sodium channel blocker. In one embodiment, either one or both of the late sodium channel blocker or the potassium channel blocker are administered in an effective amount. The two agents may be administered separately or together in separate or a combined dosage unit. If administered separately, the late sodium channel blocker may be administered before or after administration of the potassium channel blocker but typically the late sodium channel blocker will be administered prior to the potassium channel blocker.

Accordingly, in one embodiment, the invention is directed to a method for modulating ventricular and/or atrial rate in a human patient in need thereof, said method comprising administering to the patient effective amounts of one or more potassium channel blocker and one or more late sodium channel blocker. In one embodiment, the AV conduction is slowed when atrial rate is high, such as for example above 400 beats per minute or 600 beats per minute. It is contemplated that this may be beneficial to provide control of the ventricular rate during atrial fibrillation. In another embodiment, the atrial rate is decreased. In still another embodiment, the heart rate is not significantly decreased (i.e., the heart rate is not decreased by more than 2%, 5%, 10%, or no more than about 20%) during sinus rhythm.

In another embodiment, is provided a method for modulating ventricular and/or atrial rhythm in a human patient in need thereof, said method comprising administering to the patient therapeutic amounts of a potassium channel blocker and a late sodium channel blocker. In one embodiment, the sinus rhythm of the patient is maintained.

In still another embodiment, is provided a method for providing rhythm and rate control of the ventricles and/or atria in a human patient in need thereof, said method comprising administering to the patient effective amounts of one or more potassium channel blocker and one or more late sodium channel blocker.

In one embodiment, the invention is directed to a method for reducing or preventing torsades de pointes ventricular tachycardia in a human patient in need thereof, said method comprising administering to the patient effective amounts of one or more potassium channel blocker and one or more late sodium channel blocker.

It is contemplated that by preventing atrial fibrillation, both electrical and structural remodeling are modulated. This is because atrial fibrillation begets further atrial fibrillation, and fibrillation begets structural remodeling. The control provided by late sodium channel blocker and potassium channel blocker of atrial rhythm (i.e., rhythm control) will prevent the progression of atrial tachyarrhythmias from occasional self-terminated episodes to permanent AF with electrical and structural remodeling. Further, reduction of atrial rate and Na/Ca loading is expected to reduce oxidative stress and decrease cell death, reduce inflammation, and limit fibrosis (Van Wagoner D., J Cardiovasc Pharm 52: 306-313, 2008). Accordingly, the invention is also directed to a method for modulating electrical and structural remodeling in a patient in need thereof, said method comprising administering to the patient effective amounts of one or more potassium channel blocker and one or more late sodium channel blocker.

It is also contemplated that by combining late sodium channel blocker and potassium channel blocker any undesired side effects may be reduced. For example, administration of a late sodium channel blocker to a patient already receiving a potassium channel blocker therapy reduces the side effects of potassium channel blocker. The synergistic effect of combined administration will allow for a reduction in the amount of potassium channel blocker necessary to achieve a therapeutic effect, thereby resulting in a reduced incidence of undesirable side effects. As such, in one embodiment, the invention is directed to a method for reducing the undesirable side effects of a potassium channel blocker comprising administering an effective amount of a late sodium channel blocker. The reverse combination is also contemplated.

Additionally, it is contemplated that by administration of a potassium channel blocker to a human patient on a late sodium channel blocker reduces the prolongation of the QT interval. Accordingly, in one embodiment, the invention is directed to a method for reducing the prolongation of the QT interval in a patient caused by a late sodium channel blocker, said method comprising administering to the patient an effective amount of a potassium channel blocker or salt or salts thereof. In the reciprocal, a potassium channel blocker may also cause prolongation of the QT interval and as such, by administering potassium channel blocker with a late sodium channel blocker, it is contemplated a reduction of the QT interval will be seen.

As discussed above, it is contemplated that by administration of late sodium channel blocker, the therapeutically effective amount of potassium channel blocker is reduced. As such, the invention, in one embodiment, is directed to a method for reducing the therapeutically effective dose of potassium channel blocker comprising administering an effective amount of a late sodium channel blocker.

The disclosure is also directed to a method of treating or preventing supraventricular tachyarrhythmia or ventricular tachyarrhythmia in a human patient in need thereof comprising coadministering a therapeutic amount of a potassium channel blocker and equal to a therapeutic amount late sodium channel blocker.

Additionally, it is contemplated that the combination therapy reduces ventricular fibrillation in addition to atrial fibrillation. Thus, in one embodiment, the invention is directed to a method of preventing ventricular fibrillation in human patients susceptible to ventricular fibrillation, said method comprising administering to the patient effective amounts of one or more potassium channel blocker and one or more late sodium channel blocker.

As mentioned above, prolonged atrial fibrillation often results in development of congestive heart failure and/or stroke. In addition, patients with atrial fibrillation have increased risks of hospitalization and death. Thus, as a consequence of treating and preventing atrial fibrillation and ventricular arrhythmia, the combination therapy is expected to reduce hospitalization and death, the development of heart failure, and incidence of stroke. It is further contemplated that by reducing or preventing atrial fibrillation, emboli and blood clot formation is attenuated or reduced. Accordingly, in one aspect, the invention is directed to the method of preventing congestive heart failure and/or stroke in a human patient by administration of one or more potassium channel blocker or a salt or salts thereof and one or more late sodium channel blocker or a salt or salts thereof.

### 3. Compounds

Embodiments the present disclosure comprise compounds that function as late sodium channel blockers. As stated above, compounds known to be late sodium channel blockers and therefore useful in the methods and formulations described herein are described in US 2013/0012492; US 2013/0005706; US 2012/0289493; US 2009/0012103; US 2010/0197684; US 2009/0181986; US 2010/0113514; WO 2010/056865; US 2010/0125091; US 2010/0113449; US 2010/0113461; WO 2011/056985; US 2011/0021521; or WO 2012/003392, each of which are hereby incorporated by reference in its entirety.

Specific late sodium channel blockers contemplated for use in the methods and pharmaceutical compositions disclosed herein can be found in US 2013/0012492, such as for example, Formula I and II, below. The late sodium channel blockers of Formula I and II can be prepared according to US 2013/0012492, which is hereby incorporated by reference for all purposes in its entirety.

Accordingly, in some embodiments, the late sodium channel blocker as disclosed herein is of Formula I:

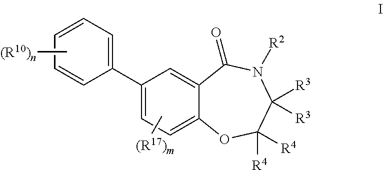

wherein:
m is 0, 1 or 2;
n is 0, 1, 2, 3, 4 or 5;
each R¹⁰ is independently selected from the group consisting of halo, —NO₂, —CN, —SF₅, —Si(CH₃)₃, —O—R²⁰, —S—R²⁰, —C(O)—R²⁰, —C(O)—OR²⁰, —N(R²⁰ (R²²), —C(O)—N(R²⁰)(R²²), —N(R²⁰)—C(O)—R²², —N(R²⁰)—C(O)—OR²², —N(R²⁰)—S(O)₂—R²⁶, —S(O)₂—R²⁰, —O—S(O)₂—R²⁰, —S(O)₂—N(R²⁰)(R²²), C₁₋₆ alkyl, C₂₋₄ alkenyl, C₂₋₄ alkynyl, cycloalkyl, aryl, heteroaryl and heterocyclyl;
   wherein said C₁₋₆ alkyl, C₂₋₄ alkenyl, C₂₋₄ alkynyl, cycloalkyl, aryl, heteroaryl or heterocyclyl are optionally substituted with one, two or three substituents independently selected from the group consisting of halo, —NO₂, aryl, heterocyclyl, heteroaryl, C₁₋₆ alkyl, C₁₋₃ haloalkyl, cycloalkyl, —N(R²⁰)(R²²), —C(O)—R²⁰, —C(O)—OR²⁰, —C(O)—N(R²⁰)(R²²), —CN and —O—R²⁰;
R² is —C₁₋₆ alkylene-R⁵, -L-R⁵, -L-C₁₋₆ alkylene-R⁵, —C₁₋₆ alkylene-L-R⁵ or —C₁₋₆ alkylene-L-C₁₋₆ alkylene-R⁵;
L is —O—, —S—, —C(O)—, —NHS(O)₂—, —S(O)₂NH—, —C(O)NH— or —NHC(O)—, provided that when R² is -L-R⁵ or -L-C₁₋₆ alkylene-R⁵, then L is not —O—, —S—, —NHS(O)₂— or —NHC(O)—;
each R³ is independently hydrogen, deuterium or C₁₋₆ alkyl optionally substituted with heteroaryl;
each R⁴ is independently hydrogen, deuterium or C₁₋₆ alkyl optionally substituted with heteroaryl;
R⁵ is cycloalkyl, aryl, heteroaryl or heterocyclyl;
   wherein said cycloalkyl, aryl, heteroaryl or heterocyclyl are optionally substituted with one, two or three substituents independently selected from the group consisting of C₁₋₆ alkyl, C₂₋₄ alkynyl, halo, —NO₂, cycloalkyl, aryl, heterocyclyl, heteroaryl, —N(R²⁰)(R²²), —N(R²⁰)—S(O)₂—R²⁰, —N(R²⁰)—C(O)—R²², —C(O)—R²⁰, —C(O)—OR²⁰, —C(O)—N(R²⁰)(R²²), —CN, oxo and —O—R²⁰;
      wherein said C₁₋₆ alkyl, cycloalkyl, aryl, heterocyclyl or heteroaryl are optionally further substituted with one, two or three substituents independently selected from the group consisting of halo, —NO₂, C₁₋₆ alkyl, cycloalkyl, aryl, heterocyclyl, heteroaryl, —N(R²⁰)(R²²), —C(O)—R²⁰, —C(O)—OR²⁰, —C(O)—N(R²⁰)(R²²), —CN and —O—R²⁰; and
         wherein said C₁₋₆ alkyl, cycloalkyl, aryl, heterocyclyl or heteroaryl are optionally further substituted with one, two or three substituents independently selected from the group consisting of halo, aryl, —NO₂, —CF₃, —N(R²⁰)(R²²), —C(O)—R²⁰, —C(O)—OR²⁰, —(O)—N(R²⁰)(R²²), —CN, —S(O)₂—R²⁰ and —O—R²⁰;
R¹⁷ is halo, —O—R²⁰ or C₁₋₆ alkyl;
R²⁰ and R²² are in each instance independently selected from the group consisting of hydrogen, C₁₋₆ alkyl, C₂₋₆ alkenyl, C₂₋₆ alkynyl, cycloalkyl, heterocyclyl, aryl and heteroaryl;
   wherein the C₁₋₆ alkyl, C₂₋₆ alkenyl, C₂₋₆ alkynyl, cycloalkyl, heterocyclyl, aryl and heteroaryl are optionally substituted with one, two or three substituents independently selected from the group consisting of hydroxyl, halo, C₁₋₄ alkyl, acylamino, oxo, —NO₂, —S(O)₂R²⁶, —CN, C₁₋₃ alkoxy, —CF₃, —OCF₃, —OCH₂CF₃, —C(O)—NH₂, aryl, cycloalkyl and heteroaryl; and
      wherein said heteroaryl is optionally further substituted with C₁₋₄ alkyl or cycloalkyl; or
when R²⁰ and R²² are attached to a common nitrogen atom R²⁰ and R²² may join to form a heterocyclic or heteroaryl ring which is then optionally substituted with one, two or three substituents independently selected from the group consisting of hydroxyl, halo, C₁₋₄ alkyl, aralkyl, aryloxy, aralkyloxy, acylamino, —NO₂, —S(O)₂R²⁶, —CN, C₁₋₃ alkoxy, —CF₃, —OCF₃, aryl, heteroaryl and cycloalkyl; and
each R²⁶ is independently selected from the group consisting of hydrogen, C₁₋₄ alkyl, aryl and cycloalkyl;
   wherein the C₁₋₄ alkyl, aryl and cycloalkyl may be further substituted with from 1 to 3 substituents independently selected from the group consisting of hydroxyl, halo, C₁₋₄ alkoxy, —CF₃ and —OCF₃;
or a pharmaceutically acceptable salt, ester, stereoisomer, mixture of stereoisomers or tautomer thereof.

In certain embodiments, the late sodium channel blocker as disclosed herein is of Formula II:

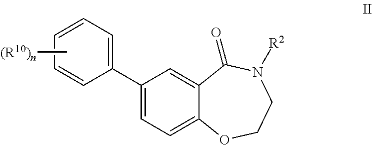

wherein:
n is 0, 1, 2 or 3;
each R¹⁰ is independently selected from the group consisting of halo, —NO₂, —CN, —SF_{S}, —Si(CH₃)₃, —O—R²⁰, —S—R²⁰, —C(O)—R²⁰, —C(O)—OR²⁰, —N(R²⁰)(R²²), —C(O)—) N(R²⁰)(R²²), —N(R²⁰)—C(O)—R²², —N(R²⁰)—C(O)—OR²², —N(R²⁰)—S(O)₂—R²⁶, —S(O)₂—R²⁰, —O—S(O)₂—R²⁰, —S(O)₂—N(R²⁰)(R²²), C₁₋₆ alkyl, C₂₋₄ alkenyl, C₂₋₄ alkynyl, cycloalkyl, aryl, heteroaryl and heterocyclyl;
   wherein said C₁₋₆ alkyl, C₂₋₄ alkenyl, C₂₋₄ alkynyl, cycloalkyl, aryl, heteroaryl or heterocyclyl are optionally substituted with one, two or three substituents independently selected from the group consisting of halo, —NO₂, aryl, heterocyclyl, heteroaryl, C₁₋₆ alkyl, C₁₋₃ haloalkyl, cycloalkyl, —N(R²⁰ (R²²), —C(O)—R²⁰, —C(O)—OR²⁰, —C(O)—N(R²⁰)(R²²), —CN and —O—R²⁰;
R² is —C₁₋₆ alkylene-R⁵;
R⁵ is cycloalkyl, aryl, heteroaryl or heterocyclyl;
   wherein said cycloalkyl, aryl, heteroaryl or heterocyclyl are optionally substituted with one, two or three substituents independently selected from the group consisting of C₁₋₆ alkyl, C₂₋₄ alkynyl, halo, —NO₂, —N(R²⁰)(R²²), —N(R²⁰)—S(O)₂—R²⁰, —N(R²⁰)—C(O)—R²², —C(O)—R²⁰, —C(O)—OR²⁰, —C(O)—N(R²⁰)(R²²), —CN, oxo and —O—R²⁰; and
      wherein said C₁₋₆ alkyl is optionally further substituted with one, two or three substituents independently selected from the group consisting of halo, —NO₂, —N(R²⁰)(R²²), —C(O)—R²⁰, —C(O)—OR²⁰, —C(O)—N(R²⁰)(R²²), —CN and —O—R²⁰;
R²⁰ and R²² are in each instance independently selected from the group consisting of hydrogen, C₁₋₆ alkyl, C₂₋₆ alkenyl, C₂₋₆ alkynyl, cycloalkyl, heterocyclyl, aryl and heteroaryl; and
   wherein the C₁₋₆ alkyl, C₂₋₆ alkenyl, C₂₋₆ alkynyl, cycloalkyl, heterocyclyl, aryl and heteroaryl are optionally substituted with one, two or three substituents independently selected from the group consisting of hydroxyl, halo, C₁₋₄ alkyl, acylamino, oxo, —NO₂, —S(O)₂R²⁶, —CN, C₁₋₃ alkoxy, —CF₃, —OCF₃, —OCH₂CF₃ and —C(O)—NH₂; or
when R²⁰ and R²² are attached to a common nitrogen atom R²⁰ and R²² may join to form a heterocyclic or heteroaryl ring which is then optionally substituted with one, two or three substituents independently selected from the group consisting of hydroxyl, halo, C₁₋₄ alkyl, aralkyl, aryloxy, aralkyloxy, acylamino, —NO₂, —S(O)₂R²⁶, —CN, C₁₋₃ alkoxy, —CF₃ and —OCF₃; and
each R²⁶ is independently selected from the group consisting of hydrogen, C₁₋₄ alkyl, aryl and cycloalkyl;
   wherein the C₁₋₄ alkyl, aryl and cycloalkyl may be further substituted with from 1 to 3 substituents independently selected from the group consisting of hydroxyl, halo, C₁₋₄ alkoxy, —CF₃ and —OCF₃;
or a pharmaceutically acceptable salt, ester, stereoisomer, mixture of stereoisomers or tautomer thereof.

In one embodiment, the late sodium channel blocker is selected from the group consisting of:
4-(pyrimidin-2-ylmethyl)-7-(4-(trifluoromethoxy)phenyl)-3,4-dihydrobenzo[f][1,4]oxazepin-5(2H)-one,
4-(pyrimidin-2-ylmethyl)-7-(4-(trifluoromethyl)phenyl)-3,4-dihydrobenzo[f][1,4]oxazepin-5(2H)-one,7-(4-chlorophenyl)-4-(pyrimidin-2-ylmethyl)-3,4-dihydrobenzo[f][1,4]oxazepin-5(2H)-one,
7-(4-tert-butylphenyl)-4-(pyrimidin-2-ylmethyl)-3,4-dihydrobenzo[f][1,4]oxazepin-5(2H)-one, and
4-(imidazo[1,2-a]pyridin-2-ylmethyl)-7-(4-(trifluoromethyl)phenyl)-3,4-dihydrobenzo[f][1,4]oxazepin-5(2H)-one,

or a pharmaceutically acceptable salt thereof.

Specific potassium channel blockers contemplated for use in the methods and pharmaceutical compositions disclosed herein, include for example, a potassium channel blocker is selected from the group consisting of bretylium, sotalol, ibutilide, dofetilide, azimilide, bretylium clofilium, E-4031, nifekalant, tedisamil, sematilide, fampridine and tertiapin.

Other potassium channel blockers contemplated for use in the methods and pharmaceutical compositions disclosed herein can be found in U.S. Pat. No. 7,456,187, such as for example, Formula III, below. The potassium channel blockers of Formula III can be prepared according to U.S. Pat. No. 7,456,187, which is hereby incorporated by reference for all purposes in its entirety.

Accordingly, in some embodiments, the late sodium channel blocker as disclosed herein is of Formula III:

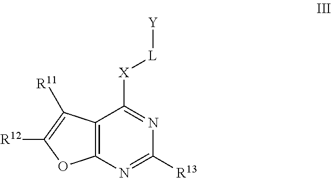

wherein:
R¹¹ is aryl, heteroaryl, cycloalkyl or alkyl;
R¹² is H, H alkyl, nitro, —CO₂R¹⁷, CONR¹⁴R¹⁵ or halo;
R¹³ is H, NR¹⁴R¹⁵, NR¹⁶C(O)R¹⁸, halo, trifluoromethyl, alkyl, cyano or alkoxy;
R¹⁴ and R¹⁵ may be the same or different, and may be H, alkyl, aryl, heteroaryl or cycloalkyl; or R¹⁴ and R¹⁵ may together form a saturated, unsaturated or partially saturated 4 to 7 member ring, wherein said ring may optionally comprise one or more further heteroatoms selected from N, O or S; X is O, S or NR¹⁶;
R¹⁶ is H or alkyl;
R¹⁷ is hydrogen, methyl or ethyl;
R¹⁸ is methyl or ethyl;
L is —(CH₂)n′—, where n′ is 1, 2 or 3; and
Y is aryl, a heterocyclic group, alkyl, alkenyl or cycloalkyl; or a pharmaceutically acceptable salt thereof

### 4. Dosing

For all of the methods described herein, it is contemplated that at least one of either late sodium channel blocker or potassium channel blocker is administered in a less than standard effective dose which becomes effective as a consequence of its administration with the other drug. However, it is also contemplated that potassium channel blocker and late sodium channel blocker may also both be administered in the standard effective amount. In some embodiments, the potassium channel blocker is administered in an effective dose and late sodium channel blocker is administered in a standard effective dose. In other embodiment, late sodium channel blocker is administered in a less than standard effective dose and potassium channel blocker is administered in an effective dose. In still other embodiments, both late sodium channel blocker and potassium channel blocker are administered in less than standard effective doses. The expression “effective amounts of potassium channel blocker and late sodium channel blocker” is intended to encompass all possible combinations of standard and less than standard effective doses of late sodium channel blocker and potassium channel blocker and their pharmaceutically acceptable salts when administered alone.

In some embodiments, potassium channel blocker and late sodium channel blocker are administered separately.

Late sodium channel blocker and potassium channel blocker may be given to the patient in either single or multiple doses by any of the accepted modes of administration of agents having similar utilities, for example as described in those patents and patent applications incorporated by reference, including buccal, by intra-arterial injection, intravenously, intraperitoneally, parenterally, intramuscularly, subcutaneously, orally, or via an impregnated or coated device such as a stent, for example, or an artery-inserted cylindrical polymer. In one embodiment, late sodium channel blocker and potassium channel blocker are administered intravenously.

In one embodiment, late sodium channel blocker and potassium channel blocker are administered orally. Potassium channel blocker and late sodium channel blocker may also be administered as a combined dosage unit, such as, for example, in a tablet.

### 5. Pharmaceutical Formulations

As mentioned above, potassium channel blocker and a late sodium channel blocker may be administered to a patient in a manner such that the two active ingredients may be formulated separately but administered at similar times (i.e., either together or one after the other). Administered also means that potassium channel blocker and late sodium channel blocker may be co-formulated into a combined dosage unit. Accordingly, in one embodiment, the invention is directed to pharmaceutical formulations comprising an effective amount of a potassium channel blocker, an effective amount of a late sodium channel blocker, and a pharmaceutically acceptable carrier.

In another embodiment, the formulation comprises a an effective amount of a late sodium channel blocker and/or a potassium channel blocker. In certain embodiments, the formulations are formulated for either intravenous or oral administration. In still other embodiment, the two active ingredients are co-formulated into a combined dosage unit. In still yet other embodiments, the two active ingredients are formulated separately for administration.

### 6. Coformulations

In certain embodiments of the present invention, the late sodium channel blocker and potassium channel blocker are coformulated into a combined dosage unit or unitary dosage form suitable for oral administration. In certain embodiments, the late sodium channel blocker is formulated as a sustained release formulation or an immediate release formulation. In certain embodiments, the potassium channel blocker is formulated for immediate release or sustained release.

### 7. Additional Formulations

Formulations also contemplated by the present invention may also be for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles. Aqueous solutions in saline are also conventionally used for injection, but less preferred in the context of the present invention. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. The same formulations are contemplated for separate administration of the late sodium channel blocker and the potassium channel blocker.

Sterile injectable solutions are prepared by incorporating the component in the required amount in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The ideal forms of the apparatus for administration of the novel combinations for atrial fibrillation and other methods of the invention consist therefore of (1) either a syringe comprising 2 compartments containing the 2 active substances ready for use or (2) a kit containing two syringes ready for use.

In making a pharmaceutical compositions that include a late sodium channel blocker and a potassium channel blocker, the active ingredients are usually diluted by an excipient or carrier and/or enclosed within such a carrier that can be in the form of a capsule, sachet, paper or other container. When the excipient serves as a diluent, in can be a solid, semi-solid, or liquid material (as above), which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compounds, soft and hard gelatin capsules, sterile injectable solutions, and sterile packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents.

The compositions of the invention can be formulated so as to provide quick, sustained or delayed release of the active ingredient after administration to the patient by employing procedures known in the art. As discussed above, given the reduced bioavailabity of late sodium channel blocker, sustained release formulations are generally preferred. Controlled release drug delivery systems for oral administration include osmotic pump systems and dissolutional systems containing polymer-coated reservoirs or drug-polymer matrix formulations. Examples of controlled release systems are given in U.S. Pat. Nos. 3,845,770; 4,326,525; 4,902,514; and 5,616,345.

The compositions are preferably formulated in a unit dosage form. The term “unit dosage forms” or “combined dosage unit” refers to physically discrete units suitable as unitary dosages for human subjects and other mammals, each unit containing a predetermined quantity of the active materials calculated to produce the desired effect, in association with a suitable pharmaceutical excipient (e.g., a tablet, capsule, ampoule). The active agents of the invention are effective over a wide dosage range and are generally administered in an effective amount. It will be understood, however, that the amount of each active agent actually administered will be determined by a physician, in the light of the relevant circumstances, including the condition to be treated, the chosen route of administration, the actual compounds administered and their relative activity, the age, weight, and response of the individual patient, the severity of the patient's symptoms, and the like.

For preparing solid compositions such as tablets, the principal active ingredients are mixed with a pharmaceutical excipient to form a solid preformulation composition containing a homogeneous mixture of a compound of the present invention. When referring to these preformulation compositions as homogeneous, it is meant that the active ingredients are dispersed evenly throughout the composition so that the composition may be readily subdivided into equally effective unit dosage forms such as tablets, pills and capsules.

The tablets or pills of the present invention may be coated or otherwise compounded to provide a dosage form affording the advantage of prolonged action, or to protect from the acid conditions of the stomach. For example, the tablet or pill can comprise an inner dosage and an outer dosage element, the latter being in the form of an envelope over the former. Late sodium channel blocker and the co-administered agent(s) can be separated by an enteric layer that serves to resist disintegration in the stomach and permit the inner element to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

Additional embodiments of the invention include kits comprising an effective amount of a late sodium channel blocker and an effective amount of a potassium channel blocker.

Activity testing is conducted in the Examples below using methods described herein and those well known in the art.

### EXAMPLES

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### LIST OF ABBREVIATIONS AND ACRONYMS

Abbreviation Meaning
° C. Degree Celsius
ACN Acetonitrile
ATP Adenosine-5′-triphosphate
ATX II Anemonia sulcata toxin
BSA Bovine Serum Albumin
CHO Chinese hamster ovary
cm Centimeter
conc Concentrated
d Doublet
DCM Dichloromethane
dd Doublet of doublets
DMEM Dulbecco's Modified Eagle Medium
DMF Dimethylformamide
DMSO/dmso Dimethylsulfoxide
dppf 1,1′-Bis(diphenylphosphino)ferrocene
ECF Extracellular fluid
EDTA Ethylenediaminetetraacetic acid
EGTA Ethylene glycol tetraacetic acid
eq Equivalents
ERP Effective refractory period
ESI Electrospray ionization
Et Ethyl
FCS Fetal Calf Serum
g Grams
h Hours
HBSS Hank's Balanced Salt solution
HEPES (4-(2-Hydroxyethyl)-1-piperazineethanesulfonic acid)
hERG human Ether-à-go-go Related Gene
HPLC High-performance liquid chromatography
Hz Hertz
i.m. Intramuscular
i.v. Intravenous
IMR-32 Human neuroblastoma cell line
iPr iso-Propyl
IU International Units
J Coupling constant
Kg Kilogram
kHz Kilohertz
LC Liquid chromatography
M Molar
m multiplet
m/z mass-to-charge ratio
M+ Mass peak
M+H Mass peak plus hydrogen
mg Milligram
MHz Megahertz
min/m Minute
ml/mL Milliliter
mM Millimolar
mm Millimeter
mmol Millimole
mol Mole
MS Mass spectroscopy
ms Millisecond
mV Millivolt
MSΩ MegaOhm
N Normal
nM Nanomolar
NMR Nuclear magnetic resonance
OD Outer diameter
prep Preparative
RT/rt Room temperature
s Second
s Singlet
SEM Standard error of the mean
t Triplet
TB Tonic Block
TFA Trifluoroacetic acid
THF Tetrahydrofuran
TLC Thin layer chromatography
UDB Use Dependent Block
δ Chemical shift
μA Microamps
μg Microgram
μL/μl Microliter
μM Micromolar

### Example 1

### 4-(pyrimidin-2-ylmethyl)-7-(4-(trifluoromethoxy)phenyl)-3,4-dihydrobenzo[f][1,4]oxazepin-5(2H)-one (Compound 1)

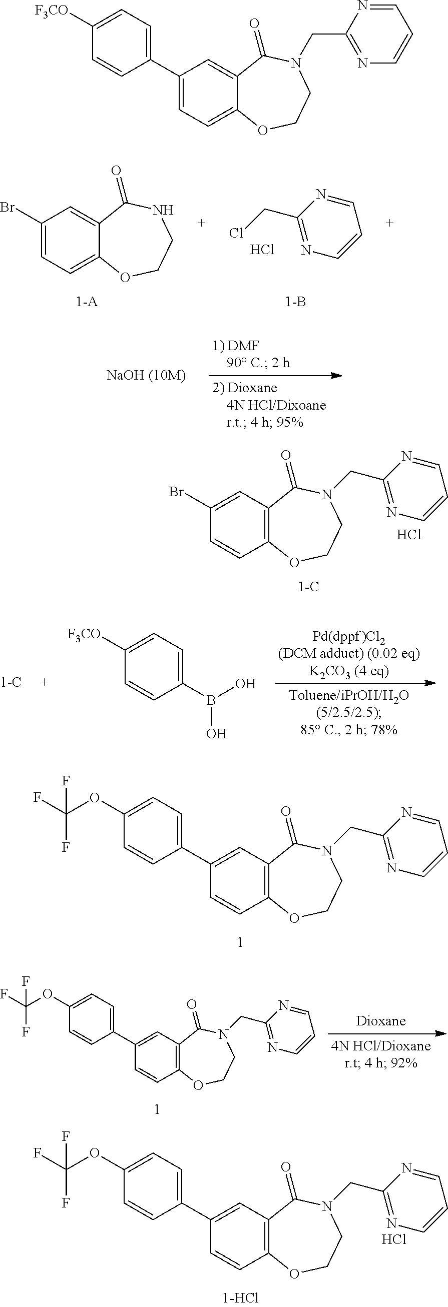

To a solution of Compound 1-A (20 g, 0.083 mol, 1 eq.) and Compound 1-B (25 g, 0.15 mol, 1.8 eq.) in DMF (150 mL), NaOH solution (20 mL, 10M, 5 eq.) was slowly added at room temperature (slightly exothermic) and stirred at r.t. for 10 min, followed by heating at 95° C. for 2 h. After cooling the reaction mixture, ethyl acetate (200 mL) was added and the organic layer was separated. The organics was washed with water (20 mL), brine, dried over sodium sulphate and concentrated.

The residue was dissolved in 1,4-dioxane (50 mL) and to this 4N HCl in dioxane (50 mL) and conc. HCl (2 mL) was added and stirred at room temperature for 4 h, filtered the precipitate, washed with ethyl acetate and dried. Compound 1-C was obtained (30 g) was a light yellow solid.

To the bromide (15 g, 0.04 mol, 1 eq), boronic acid (12.5 g, 0.06 mol, 1.5 eq) and potassium carbonate (22 g, 0.16 mol, 4 eq) in a round bottom flask, solvent (150 mL, toluene/isopropano/water:2/1/1) was added and stirred under nitrogen for 10 min. To the above solution the palladium catalyst (1 g, 0.012 mol, 0.02 eq) was added and heated at 85° C. for 2 h. The reaction mixture was diluted with ethyl acetate, separated the organic layer and filtered the organic layer through a plug of celite and silica gel and concentrated. Column purification on silica gel using ethyl acetate/hexane as eluent provided Compound 1 (13 g).

To a solution of Compound 1 (26 g) in 1,4-dioxane (25 mL), 4N HCl/dioxane (25 mL) was added followed by conc. HCl (2 mL) and stirred at room temperature for 4 h. Solvent was distilled off, dichloromethane was added and distilled off and to the residue, ethyl acetate (150 mL) was added and stirred at room temperature overnight and filtered the precipitate, washed with ethyl acetate, hexane and dried under vacuum. Compound 1-HCl obtained (24.8 g) was a white solid.

¹H-NMR (CDCl₃) δ 8.72 (d, 2H, J=5.2 Hz), 8.17 (d, 1H, J=2.4 Hz), 7.59-7.63 (m, 3H), 7.26 (d, 2H, J=3.2 Hz), 7.22 (t, 1H, J=4.8 Hz), 7.10 (d, 1H, J=8.4 Hz), 5.10 (s, 2H), 4.56 (t, 2H, J=5.0 Hz), 3.77 (t, 2H, J=5.0 Hz); MS m/z 416.1 (M+H).

### Example 2

### 4-(pyrimidin-2-ylmethyl)-7-(4-(trifluoromethyl)phenyl)-3,4-dihydrobenzo[f][1,4]oxazepin-5(2H)-one (Compound 2)

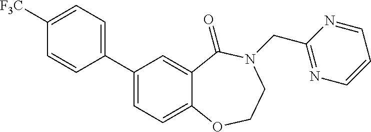 [SMILES: O=C1c2cc(-c3ccc(C(F)(F)F)cc3)ccc2OCCN1Cc1ncccn1]

Compound 2 was prepared according to Example 5 using the appropriate starting materials. ¹H-NMR (CDCl₃) δ 8.80 (d, 2H, J=5.2 Hz), 8.19 (d, 1H, J=2.8 Hz), 7.66-7.71 (m, 5H), 7.33 (t, 1H, J=5.0 Hz), 7.13 (d, 1H, J=8.4 Hz), 5.14 (s, 2H), 4.59 (t, 2H, J=4.8 Hz), 3.81 (t, 2H, J=5.0 Hz); MS m/z 400.1 (M+H).

### Example 3

### 7-(4-chlorophenyl)-4-(pyrimidin-2-ylmethyl)-3,4-dihydrobenzo[f][1,4]oxazepin-5(2H)-one (Compound 3)

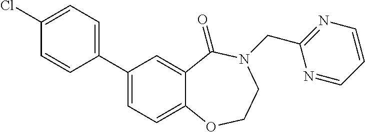 [SMILES: O=C1c2cc(-c3ccc(Cl)cc3)ccc2OCCN1Cc1ncccn1]

Compound 3 was prepared according to the Examples disclosed herein using the appropriate starting materials. MS found for C₂₀H₁₆N₃O₂Cl as (M+H)⁺ 366.1 ¹H NMR (400 MHz, dmso-d₆): δ: 8.77 (d, J=5.2 Hz, 2H), 7.94 (d, J=2.4 Hz, 1H), 7.79 (dd, J=2.4, 8.8 Hz, 1H), 7.67 (d, J=8.8 Hz, 2H), 7.49 (d, J=8.0 Hz, 2H), 7.40 (t, J=5.2 Hz, 1H), 7.13 (d, J=8.4 Hz, 1H), 4.97 (s, 2H), 4.51-4.49 (m, 2H), 3.77-3.74 (m, 2H).

### Example 4

### 7-(4-tert-butylphenyl)-4-(pyrimidin-2-ylmethyl)-3,4-dihydrobenzo[f][1,4]oxazepin-5(2H)-one (Compound 4)

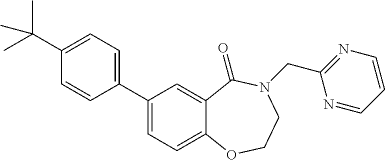 [SMILES: CC(C)(C)c1ccc(-c2ccc3c(c2)C(=O)N(Cc2ncccn2)CCO3)cc1]

Compound 4 was prepared according to the Examples disclosed herein using the appropriate starting materials. MS found for C₂₄H₂₅N₃O₃ as (M+H)⁺ 404.1 ¹H NMR (400 MHz, dmso-d₆): δ: 8.78 (d, J=5.2 Hz, 2H), 7.91 (d, J=2.4 Hz, 1H), 7.75 (dd, J=2.4, 8.8 Hz, 1H), 7.55 (d, J=8.8 Hz, 2H), 7.46 (d, J=8.0 Hz, 2H); 7.41 (t, J=5.2 Hz, 1H); 7.11 (d, J=8.4 Hz, 1H), 4.97 (s, 2H), 4.50-4.47 (m, 2H), 3.76-3.73 (m, 4H); 1.29 (s, 9H).

### Example 5

### 4-(imidazo[1,2-a]pyridin-2-ylmethyl)-7-(4-(trifluoromethyl)phenyl)-3,4-dihydrobenzo[f][1,4]oxazepin-5(2H)-one (Compound 5)

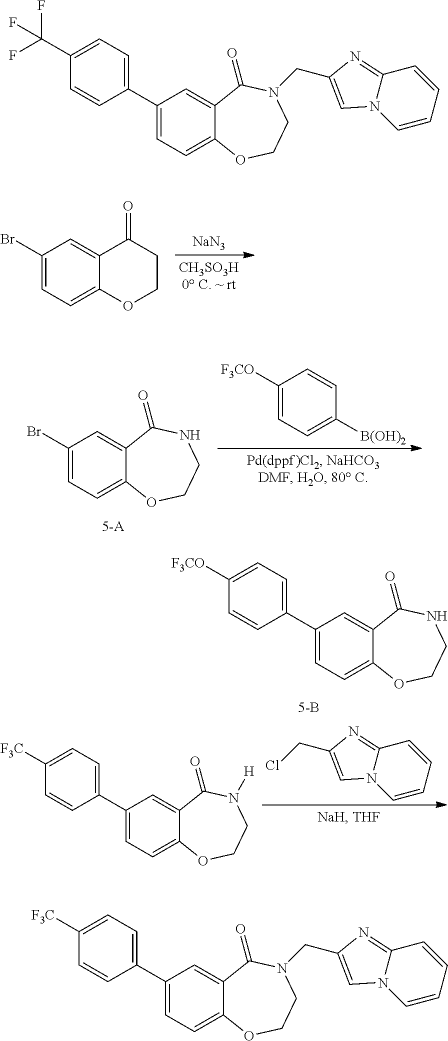

Commercially available 6-bromochroman-4-one (1.0 g, 3 mmol) was dissolved in 10 mL methanesulfonic acid. The solution was cooled using an ice bath and sodium azide (0.30 g, 4.5 mmol) was added over a period of 45 min. The mixture was stirred at RT for 16 h. The mixture was neutralized using conc. HCl. The resulting solid was filtered and washed with water to afford Compound 5-A as analytically pure sample.

For the Suzuki coupling reaction the following conditions were applied: To a suspension of Compound 5-A (1 eq), the substituted boronic acid or boronate ester (1.2 eq) and base sodium bicarbonate (3 eq) in solvent (DMF:water in the ratio of 4:1) was added palladium catalyst Pd(dppf)Cl₂ (10 mol %) and heated at 80° C. for 2-4 h. The reaction progress was followed by LC and after completion, the reaction mixture was filtered through celite, washed with ethyl acetate. The filtrate was concentrated the filtrate and purified by prep TLC/prep HPLC or column chromatography to afford Compound 5.

7-(4-(trifluoromethyl)phenyl)-3,4-dihydrobenzo[f][1,4]oxazepin-5(2H)-one (50 mg) was dissolved in dry THF and the NaH suspension (6 mg, 60% in oil) was added, followed shortly by 2-(chloromethyl)imidazo[1,2-a]pyridine (29 mg) and stirred overnight at room temperature. Worked up with ethyl acetate and pH 7 buffer organic layer dried over MgS(O)₄ and concentrated. Purification was conducted on normal phase (CH₂Cl₂/10% EtOH in ethyl acetate gradient) followed by reverse-phase (ACN/H₂O, 0.1% TFA). Resulting glassy solid was dissolved in dioxane, diluted 10-fold with 0.1N HCl and lyophilized resulting in 4-(imidazo[1,2-a]pyridin-2-ylmethyl)-7-(4-(trifluoromethyl)phenyl)-3,4-dihydrobenzo[f][1,4]oxazepin-5(2H)-one hydrochloride salt as a white solid (42.2 mg).

¹H NMR: 7.95 (s, 1H); 7.53 (d, 2H); 7.36 (m, 2H); 7.31 (d, 2H); 7.20 (d, 1H); 5.30 (s, 2H); 2.16 (s, 3H); ¹⁹F NMR: −58.36 (s); MS (ESI+): 391.0 (base peak, M+H⁺); 803.2 (2M+Na⁺).

### Example 6

### Sodium Current Screening Assays

To determine whether a compound is a late sodium channel blocker, they may be tested via the following assay.

The late sodium current (Late INa) and peak sodium current (Peak INa) assays are performed on an automated electrophysiology platform, PatchXpress 7000A (MDS Analytical Technologies, Sunnyvale, Calif.), which uses the whole cell patch clamp technique to measure currents through the cell membrane of up to 16 cells at a time. The assay uses an HEK293 (human embryonic kidney) cell line heterologously expressing the wild-type human cardiac sodium channel, hNaᵥ1.5, purchased from Millipore (Billerica, Mass.). No beta subunits were coexpressed with the Na channel alpha subunit. Cells are maintained with standard tissue culture procedures and stable channel expression is maintained with 400 μg/mL Geneticin in the culture medium. Cells isolated for use on PatchXpress are incubated for 5 minutes in Versene 1× and then for 2 minutes in 0.0125% Trypsin-EDTA (both at 37° C.) to ensure that 80-90% of the cells are single and not part of a cell cluster. Experiments are carried out at 24-27° C.

For both the Late INa and Peak INa assays, series resistance compensation is set to 50% and whole-cell compensation is performed automatically. Currents are low-pass filtered at 10 kHz and digitized at 31.25 kHz. Currents through open sodium channels are automatically recorded and stored in the DataXpress2 database (MDS Analytical Technologies, Sunnyvale, Calif.). Analysis is performed using DataXpress2 analysis software and data are compiled in Excel.

Compound stocks are routinely made in glass vials to 10 mM in dimethyl sulfoxide (DMSO). In some cases, when compounds are not soluble in DMSO, they are made in 100% ethanol. Stocks are sonicated as necessary. The extracellular solution for screening Late INa is composed of: 140 mM NaCl, 4 mM KCl, 1.8 mM CaCl₂, 0.75 mM MgCl₂, and 5 mM HEPES with pH adjusted to 7.4 using NaOH. The extracellular solution for screening Peak INa is composed of: 20 mM NaCl, 120 mM N-methyl-D glucamine, 4 mM KCl, 1.8 mM CaCl₂, 0.75 mM MgCl₂, and 5 mM HEPES with pH adjusted to 7.4 using HCl. The intracellular solution used to perfuse the inside of the cells for both the Late INa and Peak INa assays contains: 120 mM CsF, 20 mM CsCl, 5 mM EGTA, 5 mM HEPES and pH adjusted to 7.4 with CsOH. Compounds are diluted in extracellular solution to 10 μM in glass vials and then transferred to glass well plates before robotic addition to the cells. The 0Na extracellular solution used at the end of each experiment for the Late INa and Peak INa assays to measure baseline current contains: 140 mM N-methyl-D-glucamine; 4 mM KCl; 1.8 mM CaCl₂; 0.75 mM MgCl₂; 5 mM HEPES and pH was adjusted to 7.4 with HCl.

### Late INa Screening Assay

For the Late INa assay, sodium channels are activated every 10 seconds (0.1 Hz) by depolarizing the cell membrane to −20 mV for 250 milliseconds (ms) from a holding potential of −120 mV. In response to a −20 mV voltage step, typical Naᵥ1.5 sodium currents activate rapidly to a peak negative current and then inactivate nearly completely within 3-4 ms.

All compounds are tested to determine their activity in blocking the late sodium current. Late INa current is generated by adding 10 μM Tefluthrin (pyrethroid) to the extracellular solution while recording Na currents. For some experiments, 50 nM ATX II (sea anemone toxin), another late INa activator, was used to generate the late component. Both activators generate late components that are large enough that block of the late component by compounds can be measured easily. For the purposes of the screening, late INa is defined as the mean current between 225 ms and 250 ms after stepping to −20 mV to activate Na channels. After establishing the whole cell recording configuration, late INa activators are added to each well 4 times over a 16-17 minute period so that the late component of the Na current reaches a stable value. Compounds are then added (typically at 10 μM), in the presence of late INa activator, with 3 additions over the course of 7 or 8 minutes. Measurements are made typically at the end of exposure to the third compound addition. Measurements are made at the end of exposure to the third compound addition and values are normalized to the current level when all Na⁺ is removed from the extracellular solution after two additions of 0Na-ECF. Results are reported as percent block of late INa

### Peak INa Screening Assay

Compounds are also evaluated for their effect in several other assays, including their effect on Peak INa. After screening compounds against late INa, selected compounds are evaluated for their effect in several other assays, including their effect on peak INa. One goal of this program is to avoid significant block of peak INa. Since the peak INa in our cells can be very large, introducing artifacts in the recording, the concentration of Na⁺ in the bath is reduced to 20 mM and a nonpermeant cation is added to compensate for the Na⁺ that was removed to maintain the osmolarity and ionic strength of the solution (see solution details above). All measurements are normalized to the current level when all Na⁺ is removed from the extracellular solution, after two additions of 0Na-ECF.

In some cases we measured the effect of compound on peak INa using data from the late INa assay. But often peak currents are too large to make this possible, requiring that we perform a separate assay to evaluate the effect on peak INa. For the original peak INa assay the channel is activated every 10 seconds by depolarizing the cell membrane to −20 mV for 250 ms from a holding potential of −120 mV. After establishing the whole cell recording configuration, the recorded currents are allowed to stabilize for 6-7 minutes. Compound is added at 10 μM with three additions over an 8-9 minute period. Analysis of peak INa generally requires correction for rundown before determining the % block of peak current by the tested compound.

A new Peak INa screening assay was developed to allow assessment of the effect of compounds on peak INa at both low and high stimulation frequencies. The goal is to find compounds that are highly selective for block of late INa but do not block peak INa. A low stimulation frequency of 0.1 Hz is used to determine the effect of compound when the channel spends most of the time in the resting (closed) state and provides information about Tonic Block (TB). A higher stimulation frequency (3 Hz) is used to measure block of the channel when it spends more time in the activated and inactivated states, and provides a measure of Use-Dependent Block (UDB). The −100 mV holding potential and the 3 Hz stimulation frequency were chosen so that our benchmark compound would have a small but detectable effect under experimental conditions, allowing for direct comparison of new compounds with the benchmark.

For the new peak INa assay, Na⁺ channels are activated by depolarizing the cell membrane to 0 mV for 20 ms from a holding potential of −100 mV. After establishing the whole cell recording configuration, channels are stimulated to open with low frequency stimulation (0.1 Hz) for 7 minutes so that we can monitor the recording and assess the extent to which the recording has stabilized. After this stabilization period the stimulation frequency is increased to 3 Hz for 2 minutes, and then returned to 0.1 Hz. Since 3 Hz stimulation causes a small decrease in the peak current even in the absence of compound, we use this internal control for each cell, when no compound is present, to correct the results from 3 Hz stimulation when compound is present. Following 3 Hz stimulation under control conditions, the cell is allowed to recover for 200 seconds before compound is added. Compound (10 μM) is added 3 times at 60 second intervals, while stimulating the channels to open at 0.1 Hz to monitor the progression of block. After the 3^{rd} compound addition, a 320 second wait period is imposed to allow for equilibration before the second period of 3 Hz stimulation begins. TB is measured before the second period of 3 Hz stimulation. Both TB and UDB are analyzed by incorporating rundown correction for the peak INa and UDB is calculated by compensating for the small use-dependent effect of the stimulation protocol on peak INa in the absence of compound.

### Example 7

### hERG Screening Assay

Compounds are screened to test their activity in blocking the hERG potassium channel. The hERG channel is heterologously expressed in a CHO (Chinese Hamster Ovary) cell line. Cells are maintained with standard tissue culture procedures and stable channel expression is maintained with 500 μg/mL G418 reagent in the culture medium. Cells are harvested for testing on the PatchXpress automated patch clamp with Accumax (Innovative Cell Technologies, San Diego, Calif.) to isolate single cells.

The following solutions are used for electrophysiological recordings. The external solution contains: 2 mM CaCl2; 2 mM MgCl2; 4 mM KCl; 150 mM NaCl; 10 mM Glucose; 10 mM HEPES (pH 7.4 with 1M NaOH, osmolarity). The internal solution contains: 140 mM KCl, 10 mM MgCl₂, 6 mM EGTA, 5 mM HEPES, 5 mM ATP (pH adjusted to 7.25 with KOH).

hERG channels are activated when the voltage is stepped to +20 mV from the −80 mV holding potential. During a 5 second step at +20 mV, the channels activate and then largely inactivate, so the currents are relatively small. Upon returning to −50 mV from +20 mV, hERG currents transiently become much larger as inactivation is rapidly removed and then the channel closes. The first step to −50 mV for 300 ms is used as a baseline for measuring the peak amplitude during the step to −50 mV after channel activation. The peak current at −50 mV is measured both under control conditions and after addition of compound.

All compounds are prepared as 10 mM DMSO stocks in glass vials. Stock solutions are mixed by vigorous vortexing and sonication for about 2 minutes at room temperature. For testing, compounds are diluted in glass vials using an intermediate dilution step in pure DMSO and then further diluted to working concentrations in external solution. Dilutions are prepared no longer than 20 minutes before use.

After achieving the whole-cell configuration, cells are monitored for 90 seconds to assess stability and washed with external solution for 66 seconds. The voltage protocol described above is then applied to the cells every 12 seconds and throughout the whole procedure. Only cells with stable recording parameters and meeting specified health criteria are allowed to enter the compound addition procedure.

External solution containing 0.1% DMSO (vehicle) is applied to the cells first to establish the control peak current amplitude. After allowing the current to stabilize for 3 to 5 minutes, 1 μM and then 10 μM test compounds are applied. Each compound concentration is added 4 times and cells are kept in test solution until the effect of the compound reaches steady state or for a maximum of 12 minutes. After addition of test compound, a positive control (1 μM Cisapride) is added and must block >95% of the current for the experiment to be considered valid. Washout in the external solution compartment is performed until the recovery of the current reaches steady state. Data are analyzed using DataXpress, Clampfit (Molecular Devices, Inc., Sunnyvale) and Origin 7 (Originlab Corp.)

### Example 8

### L-type Calcium Channel Activity Well-Plate Assay

Cell Culture: IMR-32 (human neuroblastoma) cells were obtained from The American Type Culture Collection. The cells were maintained in MEM supplemented with 10% fetal bovine serum, 2 mM of L-glutamine, 100 IU/mL of penicillin, 50 μg/mL of streptomycin, 1% of sodium pyruvate, 1% of sodium bicarbonate and 1% of non-essential amino acid. The cells were cultured at 37° C. in a humidified 5% CO2/95% air incubator. Culture medium was changed every two days and cells were recultivated when they reached 70-80% confluent.

Assay: IMR-32 cells were seeded on a Microtest 96-well Assay Plate (BD FALCONTM) at a density of 200,000 cells/well in 200 μL culture medium for overnight. The culture medium was removed, and replaced by 120 μL Ca-4 dye (MDS Analytical Technologies, Sunnyvale, Calif.) in HBSS (1× Hank's Balanced Salt solution plus 20 mM HEPES, pH 7.4) containing 2 mM probenecid. Cells were then incubated for 1 hour at 37° C. in incubator. Testing compounds were diluted from 5 μM-50 μM in HBSS, and 40 μL were added in cells before assay. L-type calcium channel activities (Max-Min) were measured after addition of 40 μL of 1 μM (−)Bay K 8644 plus 50 mM KCl (final concentration) using FlexStation (Molecular Devices) immediately after addition of testing compounds. The inhibition of L-type calcium channel activity by compounds was then calculated.

Compounds tested and found to be effective using the described assay methods at a concentration of 1 μM and 10 μM in the late INa and Peak INa assays, and at 1 μM and 10 μM for the hERG and L-type calcium channel assays. The assay results demonstrate that the compounds tested showed activity as modulators of late sodium current, for example by inhibiting (or reducing) the late sodium current.

Compounds are tested using the described assay methods. Data are obtained by testing the listed compounds at 10 μM and 1 μM concentrations in the late INa assay, and at 1 μM and 10 μM for the hERG and L-type calcium channel assays.

### Example 9

### Kv1.5 Autopatch Electrophysiology

To test for compounds that are potassium channel blockers, the following assay may be used.

Cells stably transfected with cDNA for human Kv1.5 (in pEF6::VA-His-TOPO) are grown in Dulbecco's Modified Eagle media (DMEM) alpha supplemented with 10% Fetal Calf Serum (FCS), 20 μL/mL penicillin (5000 U/mL) streptomycin (5000 μg/mL), 10 μL/mL (100×) glutamine, and blasticidin (7.5 μg/mL).

The external bathing solution contains (in mM): 150 NaCl, 10 KCl, 100 Potassium Gluconate, 3 MgCl₂, 1 CaCl₂, 10 HEPES, pH 7.4. Patch pipettes are filled with an electrode solution of composition (in mM): 160 KCl, 0.5 MgCl₂, 10 HEPES, 1 EGTA, pH 7.4 with KOH.

Compounds are dissolved in DMSO (100%) and made up in the external bather at a concentration of 1 μM. All experiments were conducted at room temperature (22-24° C.).

A cell suspension (10 mL), with a density of 100,000 cells/mL, is aliquoted into a 15 mL centrifuge tube and transferred to an incubator (37° C., 5% CO₂) for approximately one hour before use. Following 60 min incubation, a tube is centrifuged at 1000 rpm for 4 min at room temperature. 9.5 mL supernatant is discarded, leaving a cell pellet at the bottom of the tube. The pellet is then resuspended using 100 μL of cold (4° C.), filtered (0.22 μm), 0.2% BSA/bather solution (0.02 g BSA/10 mL bather). The bottom of the tube is manually agitated gently until the solution became cloudy with cells. The 100 μL cell resuspension solution is then stored on the bench at 4° C. (using a Peltier-based temperature control device) until used.

A length of capillary glass (1B150F-4, WPI) is dipped into the cell suspension solution, such that about 3 cm column of fluid is taken up by capillary action. An Ag/AgCl wire is dropped into the non-dipped end of the capillary also. The outside of the solution-filled end of the capillary is then dried and the capillary is loaded into the AutoPatch™.

Borosilicate glass patch pipettes (from 1.5 mm OD, thin-walled filamented, GC150-TF capillary glass, Harvard) is pulled using a DMZ pipette puller (Zeitz Instruments), and are back-filled using the internal pipette solution, being careful that no bubbles remain at the tip or in the body of the pipette. Patch pipettes typically had resistances of 2.3-3.5 MΩ. Once filled, the pipette tip and a proportion of the shaft (about 15 mm) are dipped into Sigmacote (Sigma). The recording pipette is then loaded into the AutoPatch™ Automated patch-clamping was initiated by the operator, but thereafter AutoPatch.exe continues the experiment providing that pre-set conditions and criteria are satisfied.

Whole cell patch-clamp recordings are made using the AutoPatch™ rig, which incorporated an EPC9 amplifier (HEKA, Germany) under control of Pulse software (v8.54, HEKA, Germany), a motion controller with 2 translators (Newport, UK), valve controller (VF1) and a c-level suction device all at room temperature (22-24° C.). This equipment is completely under the control of AutoPatch.exe and operator intervention is only made when there is a requirement to refill the drug reservoirs or to prevent the loss of a cell due to a technical error. Cells with an Rₛₑᵣᵢₑₛ greater than 18MΩ were discounted from the experiment.

Qualification stages prior to perfusion and drug application ensured that the observed current met the criteria for the experiment. Only those cells with an I_{K}>500 were used for experiments. Cells were continuously perfused with external solution at a flow rate of 1.8-2 mL/minute. The perfusion chamber had a working volume of 80-85 μl and allowed for rapid exchange of drug solutions. Online analysis of the hKᵥ1.5 current during the application of compounds was performed by the AutoPatch™ software.

Electrophysiology voltage-step protocols and analysis of data is performed as follows. Data was sampled at 5 kHz, and filtered with a −3 dB bandwidth of 2.5 kHz. Cells are held at a voltage of −80 mV. Currents are evoked to a voltage step for 1000 ms in duration at 0 mV every 5 s. Currents are analyzed using Pulsefit software (v8.54, HEKA, Germany), with the total charge measured during the whole of the voltage step. All other plots are produced using Igor Pro (WaveMetrics).

### Example 10

### Measurement of Atrial Effective Refractory Period in Female Rabbit Isolated Heart

New Zealand White female rabbits, weighing 2.5 to 3.5 kg, were sedated then anesthetized using i.m. and i.v. injections, respectively, of xylazine and ketamine. The thorax was opened, and the heart was excised and placed in a modified Krebs-Henseleit (K-H) solution (pH 7.4, gassed with 95% O₂ and 5% CO₂). The K-H solution contained 118 mmol/l NaCl, 2.8 mmol/l KCl, 1.2 mmol/l KH₂PO₄, 2.5 mmol/l CaCl₂, 0.5 mmol/l MgSO₄, 2.0 mmol/l pyruvate, 5.5 mmol/l glucose, 0.57 mmol/l Na₂EDTA, and 25 mmol/l NaHCO₃. The aorta was cannulated, and the heart was perfused by the method of Langendorff with K-H solution warmed to 36.5° C. at a rate of 20 ml/min. A bipolar Teflon-coated electrode was placed on the right atrium to pace the heart. Electrical stimuli, 3 ms in width and 3-fold threshold amplitude, were delivered to the pacing electrode at a frequency of 1 Hz using a Grass Instruments (Quincy, Mass.) S48 stimulator.

Atrial effective refractory period (ERP) was determined using the standard extrastimulus techniques. After every eighth basic right atrial stimulus (S1S1, 300 ms), an extrastimulus (S2) was delivered with a shortening of the coupling interval (S1S2) in 10 or 2-ms steps until the S2 produced no atrial activity. ERP was defined as the longest S1S2 that failed to elicit atrial activity in response to S2.

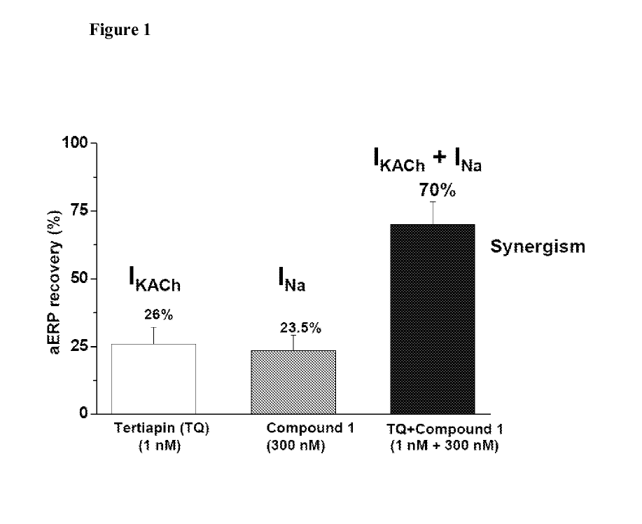

The effects of I_{KACh} blocking agent, tertiapin, and late Na⁺ current inhibitor Compound 1 on atrial ERP were evaluated in the continuous presence of 150 nM carbachol. The result can be seen in FIG. 1.

Statistical Analysis

Data are reported as mean±SEM. To compare values of measurements obtained from the same heart before and after drug treatment, repeated measures one-way analysis of variance was used, and Student-Newman-Keuls test was applied to determine which pairs of group means were significantly different. Paired and nonpaired Student t tests were used to determine the significance of a difference between two means before (as control) and after drug treatment in same or different hearts, respectively. A significant difference between two group means was defined as P<0.05.

The data has shown that coadministering a I_{KACh} blocking agent and late Na⁺ current inhibitor prolongs ERP. The synergistic effect on the rabbit atrial effective refractory period can be seen in FIG. 1.

Source of Drugs

Compound 1 was prepared by Gilead Sciences, Foster City, Calif. Carbachol and tertiapin were purchased from Sigma-Aldrich (St. Louis, Mo.).

1. A method for treating or preventing atrial fibrillation and/or atrial flutter in a human patient in need thereof comprising administering effective amounts of one or more potassium channel blocker and one or more late sodium channel blocker, wherein
the one or more potassium channel blocker is selected from the group consisting of bretylium, sotalol, ibutilide, dofetilide, azimilide, bretylium clofilium, E-4031, nifekalant, tedisamil, sematilide, fampridine and tertiapin, and
the one or more late sodium channel blocker is a compound of Formula I: 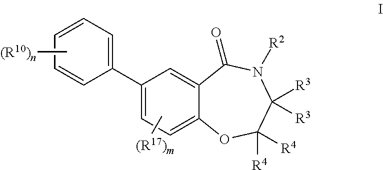
wherein:
m is 0, 1 or 2;
n is 0, 1, 2, 3, 4 or 5;
each R¹⁰ is independently selected from the group consisting of halo, —NO₂, —CN, —SF₅—Si(CH₃)₃, —O—R²⁰, —S—R²⁰, —C(O)—R²⁰, —C(O)—OR²⁰, —N(R²⁰)(R²²), —C(O)—N(R²⁰)(R²²), —N(R²⁰)—C(O)—R²², —N(R²⁰)—C(O)—OR²², —N(R²⁰)—S(O)₂—R²⁶, —S(O)₂—R²⁰, —O—S(O)₂—R²⁰, —S(O)₂—N(R²⁰)(R²²), C₁₋₆ alkyl, C₂₋₄ alkenyl, C₂₋₄ alkynyl, cycloalkyl, aryl, heteroaryl and heterocyclyl;
wherein said C₁₋₆ alkyl, C₂₋₄ alkenyl, C₂₋₄ alkynyl, cycloalkyl, aryl, heteroaryl or heterocyclyl are optionally substituted with one, two or three substituents independently selected from the group consisting of halo, —NO₂, aryl, heterocyclyl, heteroaryl, C₁₋₆ alkyl, C₁₋₃ haloalkyl, cycloalkyl, —N(R²⁰)(R²²), —C(O)—R²⁰, —C(O)—OR²⁰, —C(O)—N(R²⁰)(R²²), —CN and —O—R²⁰;
R² is —C₁₋₆ alkylene-R⁵, -L-R⁵, -L-C₁₋₆ alkylene-R⁵, —C₁₋₆ alkylene-L-R⁵ or —C₁₋₆ alkylene-L-C₁₋₆ alkylene-R⁵;
L is —O—, —S—, —C(O)—, —NHS(O)₂—, —S(O)₂NH—, —C(O)NH— or —NHC(O)—, provided that when R² is -L-R⁵ or -L-C₁₋₆ alkylene-R⁵, then L is not —O—, —S—, —NHS(O)₂— or —NHC(O)—;
each R³ is independently hydrogen, deuterium or C₁₋₆ alkyl optionally substituted with heteroaryl;
each R⁴ is independently hydrogen, deuterium or C₁₋₆ alkyl optionally substituted with heteroaryl;
R⁵ is cycloalkyl, aryl, heteroaryl or heterocyclyl;
wherein said cycloalkyl, aryl, heteroaryl or heterocyclyl are optionally substituted with one, two or three substituents independently selected from the group consisting of C₁₋₆ alkyl, C₂₋₄ alkynyl, halo, —NO₂, cycloalkyl, aryl, heterocyclyl, heteroaryl, —N(R²⁰)(R²²), —N(R²⁰)—S(O)₂—R²⁰, —N(R²⁰)—C(O)—R²², —C(O)—R²⁰, —C(O)—OR²⁰, —C(O)—N(R²⁰)(R²²), —CN, oxo and —O—R²⁰;
wherein said C₁₋₆ alkyl, cycloalkyl, aryl, heterocyclyl or heteroaryl are optionally further substituted with one, two or three substituents independently selected from the group consisting of halo, —NO₂, C₁₋₆ alkyl, cycloalkyl, aryl, heterocyclyl, heteroaryl, —N(R²⁰)(R²²), —C(O)—R²⁰, —C(O)—OR²⁰, —C(O)—N(R²⁰)(R²²), —CN and —O—R²⁰; and
wherein said C₁₋₆ alkyl, cycloalkyl, aryl, heterocyclyl or heteroaryl are optionally further substituted with one, two or three substituents independently selected from the group consisting of halo, aryl, —NO₂, —CF₃, —N(R²⁰)(R²²), —C(O)—R²⁰, —C(O)—OR²⁰, —C(O)—N(R²⁰)(R²²), —CN, —S(O)₂—R²⁰ and —O—R²⁰;
R¹⁷ is halo, —O—R²⁰ or C₁₋₆ alkyl;
R²⁰ and R²² are in each instance independently selected from the group consisting of hydrogen, C₁₋₆ alkyl, C₂₋₆ alkenyl, C₂₋₆ alkynyl, cycloalkyl, heterocyclyl, aryl and heteroaryl;
wherein the C₁₋₆ alkyl, C₂₋₆ alkenyl, C₂₋₆ alkynyl, cycloalkyl, heterocyclyl, aryl and heteroaryl are optionally substituted with one, two or three substituents independently selected from the group consisting of hydroxyl, halo, C₁₋₄ alkyl, acylamino, oxo, —NO₂, —S(O)₂R²⁶, —CN, C₁₋₃ alkoxy, —CF₃, —OCF₃, —OCH₂CF₃, —C(O)—NH₂, aryl, cycloalkyl and heteroaryl; and
wherein said heteroaryl is optionally further substituted with C₁₋₄ alkyl or cycloalkyl; or
when R²⁰ and R²² are attached to a common nitrogen atom R²⁰ and R²² may join to form a heterocyclic or heteroaryl ring which is then optionally substituted with one, two or three substituents independently selected from the group consisting of hydroxyl, halo, C₁₋₄ alkyl, aralkyl, aryloxy, aralkyloxy, acylamino, —NO₂, —S(O)₂R²⁶, —CN, C₁₋₃ alkoxy, —CF₃, —OCF₃, aryl, heteroaryl and cycloalkyl; and
each R²⁶ is independently selected from the group consisting of hydrogen, C₁₋₄ alkyl, aryl and cycloalkyl;
wherein the C₁₋₄ alkyl, aryl and cycloalkyl may be further substituted with from 1 to 3 substituents independently selected from the group consisting of hydroxyl, halo, C₁₋₄ alkoxy, —CF₃ and —OCF₃;
or a pharmaceutically acceptable salt, ester, stereoisomer, mixture of stereoisomers or tautomer thereof.

2. The method of claim 1, wherein the potassium channel blocker and the late sodium channel blocker are administered separately.

3. The method of claim 1, wherein the potassium channel blocker and the late sodium channel blocker are administered intravenously.

4. The method of claim 1, wherein the potassium channel blocker and the late sodium channel blocker are administered orally.

5. The method of claim 4, wherein the potassium channel blocker and the late sodium channel blocker are administered as a combined dosage unit.

6-26. (canceled)

27. The method of claim 1, wherein the compound is represented by Formula II: 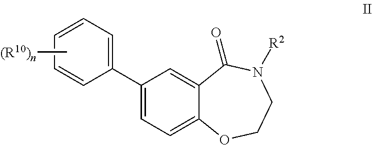
wherein:
n is 0, 1, 2 or 3;
each R¹⁰ is independently selected from the group consisting of halo, —NO₂, —CN, —SF₅, —Si(CH₃)₃, —O—R²⁰, —S—R²⁰, —C(O)—R²⁰, —C(O)—OR²⁰, —N(R²⁰)(R²²), —C(O)—N(R²⁰)(R²²), —N(R²⁰)—C(O)—R²², —N(R²⁰)—C(O)—OR²², —N(R²⁰)—S(O)₂—R²⁶, —S(O)₂—R²⁰, —O—S(O)₂—R²⁰, —S(O)₂—N(R²⁰)(R²²), C₁₋₆ alkyl, C₂₋₄ alkenyl, C₂₋₄ alkynyl, cycloalkyl, aryl, heteroaryl and heterocyclyl;
wherein said C₁₋₆ alkyl, C₂₋₄ alkenyl, C₂₋₄ alkynyl, cycloalkyl, aryl, heteroaryl or heterocyclyl are optionally substituted with one, two or three substituents independently selected from the group consisting of halo, —NO₂, aryl, heterocyclyl, heteroaryl, C₁₋₆ alkyl, C₁₋₃ haloalkyl, cycloalkyl, —N(R²⁰)(R²²), —C(O)—R²⁰, —C(O)—OR²⁰, —C(O)—N(R²⁰)(R²²), —CN and —O—R²⁰;
R² is —C₁₋₆ alkylene-R⁵;
R⁵ is cycloalkyl, aryl, heteroaryl or heterocyclyl;
wherein said cycloalkyl, aryl, heteroaryl or heterocyclyl are optionally substituted with one, two or three substituents independently selected from the group consisting of C₁₋₆ alkyl, C₂₋₄ alkynyl, halo, —NO₂, —N(R²⁰)(R²²), —N(R²⁰)—S(O)₂—R²⁰, —N(R²⁰)—C(O)—R²², —C(O)—R²⁰, —C(O)—OR²⁰, —C(O)—N(R²⁰)(R²²), —CN, oxo and —O—R²⁰; and
wherein said C₁₋₆ alkyl is optionally further substituted with one, two or three substituents independently selected from the group consisting of halo, —NO₂, —N(R²⁰)(R²²), —C(O)—R²⁰, —C(O)—OR²⁰, —C(O)—N(R²⁰)(R²²), —CN and —O—R²⁰;
R²⁰ and R²² are in each instance independently selected from the group consisting of hydrogen, C₁₋₆ alkyl, C₂₋₆ alkenyl, C₂₋₆ alkynyl, cycloalkyl, heterocyclyl, aryl and heteroaryl; and
wherein the C₁₋₆ alkyl, C₂₋₆ alkenyl, C₂₋₆ alkynyl, cycloalkyl, heterocyclyl, aryl and heteroaryl are optionally substituted with one, two or three substituents independently selected from the group consisting of hydroxyl, halo, C₁₋₄ alkyl, acylamino, oxo, —NO₂, —S(O)₂R²⁶, —CN, C₁₋₃ alkoxy, —CF₃, —OCF₃, —OCH₂CF₃ and —C(O)—NH₂; or
when R²⁰ and R²² are attached to a common nitrogen atom R²⁰ and R²² may join to form a heterocyclic or heteroaryl ring which is then optionally substituted with one, two or three substituents independently selected from the group consisting of hydroxyl, halo, C₁₋₄ alkyl, aralkyl, aryloxy, aralkyloxy, acylamino, —NO₂, —S(O)₂R²⁶, —CN, C₁₋₃ alkoxy, —CF₃ and —OCF₃; and
each R²⁶ is independently selected from the group consisting of hydrogen, C₁₋₄ alkyl, aryl and cycloalkyl;
wherein the C₁₋₄ alkyl, aryl and cycloalkyl may be further substituted with from 1 to 3 substituents independently selected from the group consisting of hydroxyl, halo, C₁₋₄ alkoxy, —CF₃ and —OCF₃;
or a pharmaceutically acceptable salt, ester, stereoisomer, mixture of stereoisomers or tautomer thereof.

28. The method of claim 27, wherein the compound is selected from the group consisting of:
4-(pyrimidin-2-ylmethyl)-7-(4-(trifluoromethoxy)phenyl)-3,4-dihydrobenzo[f][1,4]oxazepin-5(2H)-one,
4-(pyrimidin-2-ylmethyl)-7-(4-(trifluoromethyl)phenyl)-3,4-dihydrobenzo[f][1,4]oxazepin-5(2H)-one,7-(4-chlorophenyl)-4-(pyrimidin-2-ylmethyl)-3,4-dihydrobenzo[f][1,4]oxazepin-5(2H)-one,
7-(4-tert-butylphenyl)-4-(pyrimidin-2-ylmethyl)-3,4-dihydrobenzo[f][1,4]oxazepin-5(2H)-one, and
4-(imidazo[1,2-a]pyridin-2-ylmethyl)-7-(4-(trifluoromethyl)phenyl)-3,4-dihydrobenzo[f][1,4]oxazepin-5(2H)-one,
or a pharmaceutically acceptable salt thereof.

29. (canceled)

30. A pharmaceutical formulation comprising effective amounts of one or more late sodium channel blocker and one or more potassium channel blocker and a pharmaceutically acceptable carrier, wherein
the potassium channel blocker is selected from the group consisting of bretylium, sotalol, ibutilide, dofetilide, azimilide, bretylium clofilium, E-4031, nifekalant, tedisamil, sematilide, fampridine and tertiapin, and
the late sodium channel blocker is a compound of Formula I: 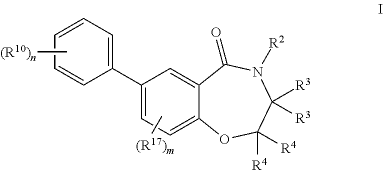
wherein:
m is 0, 1 or 2;
n is 0, 1, 2, 3, 4 or 5;
each R¹⁰ is independently selected from the group consisting of halo, —NO₂, —CN, —SF₅, —Si(CH₃))₃, —O—R²⁰, —S—R²⁰, —C(O)—R²⁰, —C(O)—OR²⁰, —N(R²⁰)(R²²), —C(O)—N(R²⁰)(R²²), —N(R²⁰)—C(O)—R²², —N(R²⁰)—C(O)—OR²², —N(R²⁰)—S(O)₂—R²⁶, —S(O)₂—R²⁰, —O—S(O)₂—R²⁰, —S(O)₂—N(R²⁰)(R²²), C₁₋₆ alkyl, C₂₋₄ alkenyl, C₂₋₄ alkynyl, cycloalkyl, aryl, heteroaryl and heterocyclyl;
wherein said C₁₋₆ alkyl, C₂₋₄ alkenyl, C₂₋₄ alkynyl, cycloalkyl, aryl, heteroaryl or heterocyclyl are optionally substituted with one, two or three substituents independently selected from the group consisting of halo, —NO₂, aryl, heterocyclyl, heteroaryl, C₁₋₆ alkyl, C₁₋₃ haloalkyl, cycloalkyl, —N(R²⁰)(R²²), —C(O)—R²⁰, —C(O)—OR²⁰, —C(O)—N(R²⁰)(R²²), —CN and —O—R²⁰;
R² is —C₁₋₆ alkylene-R⁵, -L-R⁵, -L-C₁₋₆ alkylene-R⁵, —C₁₋₆ alkylene-L-R⁵ or —C₁₋₆ alkylene-L-C₁₋₆ alkylene-R⁵;
L is —O—, —S—, —C(O)—, —NHS(O)₂—, —S(O)₂NH—, —C(O)NH— or —NHC(O)—, provided that when R² is -L-R⁵ or -L-C₁₋₆ alkylene-R⁵, then L is not —O—, —S—, —NHS(O)₂— or —NHC(O)—;
each R³ is independently hydrogen, deuterium or C₁₋₆ alkyl optionally substituted with heteroaryl;
each R⁴ is independently hydrogen, deuterium or C₁₋₆ alkyl optionally substituted with heteroaryl;
R⁵ is cycloalkyl, aryl, heteroaryl or heterocyclyl;
wherein said cycloalkyl, aryl, heteroaryl or heterocyclyl are optionally substituted with one, two or three substituents independently selected from the group consisting of C₁₋₆ alkyl, C₂₋₄ alkynyl, halo, —NO₂, cycloalkyl, aryl, heterocyclyl, heteroaryl, —N(R²⁰)(R²²), —N(R²⁰)—S(O)₂—R²⁰, —N(R²⁰)—C(O)—R²², —C(O)—R²⁰, —C(O)—OR²⁰, —C(O)—N(R²⁰)(R²²), —CN, oxo and —O—R²⁰;
wherein said C₁₋₆ alkyl, cycloalkyl, aryl, heterocyclyl or heteroaryl are optionally further substituted with one, two or three substituents independently selected from the group consisting of halo, —NO₂, C₁₋₆ alkyl, cycloalkyl, aryl, heterocyclyl, heteroaryl, —N(R²⁰)(R²²), —C(O)—R²⁰, —C(O)—OR²⁰, —C(O)—N(R²⁰)(R²²), —CN and —O—R²⁰; and
wherein said C₁₋₆ alkyl, cycloalkyl, aryl, heterocyclyl or heteroaryl are optionally further substituted with one, two or three substituents independently selected from the group consisting of halo, aryl, —NO₂, —CF₃, —N(R²⁰)(R²²), —C(O)—R²⁰, —C(O)—OR²⁰, —C(O)—N(R²⁰)(R²²), —CN, —S(O)₂—R²⁰ and —O—R²⁰;
R¹⁷ is halo, —O—R²⁰ or C₁₋₆ alkyl;
R²⁰ and R²² are in each instance independently selected from the group consisting of hydrogen, C₁₋₆ alkyl, C₂₋₆ alkenyl, C₂₋₆ alkynyl, cycloalkyl, heterocyclyl, aryl and heteroaryl;
wherein the C₁₋₆ alkyl, C₂₋₆ alkenyl, C₂₋₆ alkynyl, cycloalkyl, heterocyclyl, aryl and heteroaryl are optionally substituted with one, two or three substituents independently selected from the group consisting of hydroxyl, halo, C₁₋₄ alkyl, acylamino, oxo, —NO₂—S(O)₂R²⁶, —CN, C₁₋₃ alkoxy, —CF₃—OCF₃—OCH₂CF₃, —C(O)—NH₂, aryl, cycloalkyl and heteroaryl; and
wherein said heteroaryl is optionally further substituted with C₁₋₄ alkyl or cycloalkyl; or
when R²⁰ and R²² are attached to a common nitrogen atom R²⁰ and R²² may join to form a heterocyclic or heteroaryl ring which is then optionally substituted with one, two or three substituents independently selected from the group consisting of hydroxyl, halo, C₁₋₄ alkyl, aralkyl, aryloxy, aralkyloxy, acylamino, —NO₂—S(O)₂R²⁶, —CN, C₁₋₃ alkoxy, —CF₃, —OCF₃, aryl, heteroaryl and cycloalkyl; and
each R²⁶ is independently selected from the group consisting of hydrogen, C₁₋₄ alkyl, aryl and cycloalkyl;
wherein the C₁₋₄ alkyl, aryl and cycloalkyl may be further substituted with from 1 to 3 substituents independently selected from the group consisting of hydroxyl, halo C₁₋₄ alkoxy, —CF₃ and —OCF₃;
or a pharmaceutically acceptable salt, ester, stereoisomer, mixture of stereoisomers or tautomer thereof.

31. The pharmaceutical formulation of claim 30, formulated for intravenous administration.

32. The pharmaceutical formulation of claim 30, formulated for oral administration.

33. The pharmaceutical formulation of claim 32, wherein the formulation is in tablet form or capsule form.

34. A method for prolonging atrial effective refractory period (ERP) in a human patient in need thereof, comprising administering effective amounts of one or more potassium channel blocker and one or more late sodium channel blocker, wherein
the one or more potassium channel blocker is selected from the group consisting of bretylium, sotalol, ibutilide, dofetilide, azimilide, bretylium clofilium, E-4031, nifekalant, tedisamil, sematilide, fampridine and tertiapin, and
the one or more late sodium channel blocker is a compound of Formula I: 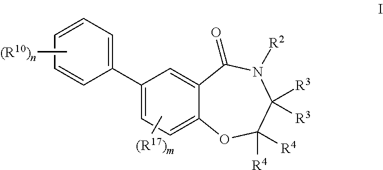
wherein:
m is 0, 1 or 2;
n is 0, 1, 2, 3, 4 or 5;
each R¹⁰ is independently selected from the group consisting of halo, —NO₂, —CN, —SF₅, —Si(CH₃)₃, —O—R²⁰, —S—R²⁰, —C(O)—R²⁰, —C(O)—OR²⁰, —N(R²⁰ (R²²), —C(O)—N(R²⁰)(R²²), —N(R²⁰)—C(O)—R²², —N(R²⁰)—C(O)—OR²², —N(R²⁰)—S(O)₂—R²⁶, —S(O)₂—R²⁰, —O—S(O)₂—R²⁰, —S(O)₂—N(R²⁰)(R²²), C₁₋₆ alkyl, C₂₋₄ alkenyl, C₂₋₄ alkynyl, cycloalkyl, aryl, heteroaryl and heterocyclyl;
wherein said C₁₋₆ alkyl, C₂₋₄ alkenyl, C₂₋₄ alkynyl, cycloalkyl, aryl, heteroaryl or heterocyclyl are optionally substituted with one, two or three substituents independently selected from the group consisting of halo, —NO₂, aryl, heterocyclyl, heteroaryl, C₁₋₆ alkyl, C₁₋₃ haloalkyl, cycloalkyl, —N(R²⁰)(R²²), —C(O)—R²⁰, —C(O)—OR²⁰, —C(O)—N(R²⁰)(R²²), —CN and —O—R²⁰;
R² is —C₁₋₆ alkylene-R⁵, -L-R⁵, -L-C₁₋₆ alkylene-R⁵, —C₁₋₆ alkylene-L-R⁵ or —C₁₋₆ alkylene-L-C₁₋₆ alkylene-R⁵;
L is —O—, —S—, —C(O)—, —NHS(O)₂—, —S(O)₂NH—, —C(O)NH— or —NHC(O)—, provided that when R² is -L-R⁵ or -L-C₁₋₆ alkylene-R⁵, then L is not —O—, —S—, —NHS(O)₂— or —NHC(O)—;
each R³ is independently hydrogen, deuterium or C₁₋₆ alkyl optionally substituted with heteroaryl;
each R⁴ is independently hydrogen, deuterium or C₁₋₆ alkyl optionally substituted with heteroaryl;
R⁵ is cycloalkyl, aryl, heteroaryl or heterocyclyl;
wherein said cycloalkyl, aryl, heteroaryl or heterocyclyl are optionally substituted with one, two or three substituents independently selected from the group consisting of C₁₋₆ alkyl, C₂₋₄ alkynyl, halo, —NO₂, cycloalkyl, aryl, heterocyclyl, heteroaryl, —N(R²⁰ (R²²), —N(R²⁰)—S(O)₂—R²⁰, —N(R²⁰)—C(O)—R²², —C(O)—R²⁰, —C(O)—OR²⁰, —C(O)—N(R²⁰)(R²²), —CN, oxo and —O—R²⁰;
wherein said C₁₋₆ alkyl, cycloalkyl, aryl, heterocyclyl or heteroaryl are optionally further substituted with one, two or three substituents independently selected from the group consisting of halo, —NO₂, C₁₋₆ alkyl, cycloalkyl, aryl, heterocyclyl, heteroaryl, —N(R²⁰)(R²²), —C(O)—R²⁰, —C(O)—OR²⁰, —C(O)—N(R²⁰)(R²²), —CN and —O—R²⁰; and
wherein said C₁₋₆ alkyl, cycloalkyl, aryl, heterocyclyl or heteroaryl are optionally further substituted with one, two or three substituents independently selected from the group consisting of halo, aryl, —NO₂, —CF₃, —N(R²⁰)(R²²), —C(O)—R²⁰, —C(O)—OR²⁰, —C(O)—N(R²⁰)(R²²), —CN, —S(O)₂—R²⁰ and —O—R²⁰;
R¹⁷ is halo, —O—R²⁰ or C₁₋₆ alkyl;
R²⁰ and R²² are in each instance independently selected from the group consisting of hydrogen, C₁₋₆ alkyl, C₂₋₆ alkenyl, C₂₋₆ alkynyl, cycloalkyl, heterocyclyl, aryl and heteroaryl;
wherein the C₁₋₆ alkyl, C₂₋₆ alkenyl, C₂₋₆ alkynyl, cycloalkyl, heterocyclyl, aryl and heteroaryl are optionally substituted with one, two or three substituents independently selected from the group consisting of hydroxyl, halo, C₁₋₄ alkyl, acylamino, oxo, —NO₂, —S(O)₂R²⁶, —CN, C₁₋₃ alkoxy, —CF₃, —OCF₃, —OCH₂CF₃, —C(O)—NH₂, aryl, cycloalkyl and heteroaryl; and
wherein said heteroaryl is optionally further substituted with C₁₋₄ alkyl or cycloalkyl; or
when R²⁰ and R²² are attached to a common nitrogen atom R²⁰ and R²² may join to form a heterocyclic or heteroaryl ring which is then optionally substituted with one, two or three substituents independently selected from the group consisting of hydroxyl, halo, C₁₋₄ alkyl, aralkyl, aryloxy, aralkyloxy, acylamino, —NO₂, —S(O)₂R²⁶, —CN, C₁₋₃ alkoxy, —CF₃, —OCF₃, aryl, heteroaryl and cycloalkyl; and
each R²⁶ is independently selected from the group consisting of hydrogen, C₁₋₄ alkyl, aryl and cycloalkyl;
wherein the C₁₋₄ alkyl, aryl and cycloalkyl may be further substituted with from 1 to 3 substituents independently selected from the group consisting of hydroxyl, halo, C₁₋₄ alkoxy, —CF₃ and —OCF₃;
or a pharmaceutically acceptable salt, ester, stereoisomer, mixture of stereoisomers or tautomer thereof.

35. The method of claim 34, wherein the patient suffers from ventricular arrhythmia.

36. The method of claim 34, wherein the patient suffers from atrial fibrillation and/or flutter.
